SEQUENCE LISTING (14 sequences)

SEQ ID NO: 1 - DNA, 14773 bp, Homo sapiens
ctgggcggccgggcgcggggagagggcgcgggagcggctcgtgcggcaggtaccatgcggacgcgcgagcccggcgagggccccggcaggcccggtccctgctcgggggcgcgctgagacggcgggtgagctccacgagagcgccgtcgccacttcgggccaactttgcgattcccgacagttaagcaatggggagacatttggctttgctcctgcttctgctccttctcttccaacattttggagacagtgatggcagccaacgacttgaacagactcctctgcagtttacacacctcgagtacaacgtcaccgtgcaggagaactctgcagctaagacttatgtggggcatcctgtcaagatgggtgtttacattacacatccagcgtgggaagtaaggtacaaaattgtttccggagacagtgaaaacctgttcaaagctgaagagtacattctcggagacttttgctttctaagaataaggaccaaaggaggaaatacagctattcttaatagagaagtgaaggatcactacacattgatagtgaaagcacttgaaaaaaatactaatgtggaggcgcgaacaaaggtcagggtgcaggtgctggatacaaatgacttgagaccgttattctcacccacctcatacagcgtttctttacctgaaaacacagctataaggaccagtatcgcaagagtcagcgccacggatgcagacataggaaccaacggggaattttactacagttttaaagatcgaacagatatgtttgctattcacccaaccagtggtgtgatagtgttaactggtagacttgattacctagagaccaagctctatgagatggaaatcctcgctgcggaccgtggcatgaagttgtatgggagcagtggcatcagcagcatggccaagctaacggtgcacatcgaacaggccaatgaatgtgctccggtgataacagcagtgacattgtcaccatcagaactggacagggacccagcatatgcaattgtgacagtggatgactgcgatcagggtgccaatggtgacatagcatctttaagcatcgtggcaggtgaccttctccagcagtttagaacagtgaggtcctttccagggagtaaggagtataaagtcaaagccatcggtggcattgattgggacagtcatcctttcggctacaatctcacactacaggctaaagataaaggaactccgccccagttctcttctgttaaagtcattcacgtgacttctccacagttcaaagccgggccagtcaagtttgaaaaggatgtttacagagcagaaataagtgaatttgctcctcccaacacacctgtggtcatggtaaaggccattcctgcttattcccatttgaggtatgtttttaaaagtacacctggaaaagctaaattcagtttaaattacaacactggtctcatttctattttagaaccagttaaaagacagcaggcagcccattttgaacttgaagtaacaacaagtgacagaaaagcgtccaccaaggtcttggtgaaagtcttaggtgcaaatagcaatccccctgaatttacccagacagcgtacaaagctgcttttgatgagaacgtgcccattggtactactgtcatgagcctgagtgccgtagaccctgatgagggtgagaacgggtacgtgacatacagtatcgcaaatttaaatcatgtgccgtttgcgattgaccatttcactggtgccgtgagtacgtcagaaaacctggactacgaactgatgcctcgggtttatactctgaggattcgtgcatcagactggggcttgccgtaccgccgggaagtcgaagtccttgctacaattactctcaataacttgaatgacaacacacctttgtttgagaaaataaattgtgaagggacaattcccagagatctaggcgtgggagagcaaataaccactgtttctgctattgatgcagatgaacttcagttggtacagtatcagattgaagctggaaatgaactggatttctttagtttaaaccccaactcgggggtattgtcattaaagcgatcgctaatggatggcttaggtgcaaaggtgtctttccacagtctgagaatcacagctacagatggagaaaattttgccacaccattatatatcaacataacagtggctgccagtcacaagctggtaaacttgcagtgtgaagagactggtgttgccaaaatgctggcagagaagctcctgcaggcaaataaattacacaaccagggagaggtggaggatattttcttcgattctcactctgtcaatgctcacataccgcagtttagaagcactcttccgactggtattcaggtaaaggaaaaccagcctgtgggttccagtgtaattttcatgaactccactgaccttgacactggcttcaatggaaaactggtctatgctgtttctggaggaaatgaggatagttgcttcatgattgatatggaaacaggaatgctgaaaattttatctcctcttgaccgtgaaacaacagacaaatacaccctgaatattaccgtctatgaccttgggataccccagaaggctgcgtggcgtcttctacatgtcgtggttgtcgatgccaatgataatccacccgagtttttacaggagagctattttgtggaagtgagtgaagacaaggaggtacatagtgaaatcatccaggttgaagccacagataaagacctggggcccaacggacacgtgacgtactcaattgttacagacacagacacattttcaattgacagcgtgacgggtgttgttaacatcgcacgccctctggatcgagagctgcagcatgagcactccttaaagattgaggccagggaccaagccagagaagagcctcagctgttctccactgtcgttgtgaaagtatcactagaagatgttaatgacaacccacctacatttattccacctaattatcgtgtgaaagtccgagaggatcttccagaaggaaccgtcatcatgtggttagaagcccacgatcctgatttaggtcagtctggtcaggtgagatacagccttctggaccacggagaaggaaacttcgatgtggataaactcagtggagcagttaggatcgtccagcagttggactttgagaagaagcaagtgtataatctcactgtgagggccaaagacaagggaaagccagtttctctgtcttctacttgctatgttgaagttgaggtggttgatgtgaatgagaacctgcacccacccgtgttttccagctttgtggaaaaggggacagtgaaagaagatgcacctgttggttcattggtaatgacggtgtcggctcatgatgaggacgccagaagagatggggagatccgatactccattagagatggctctggcgttggtgttttcaaaataggtgaagagacaggtgtcatagagacgtcagatcgactggaccgtgaatcgacctcccattattggctaacagtctttgcaaccgatcagggtgtcgtgcctctttcatcgttcatagagatctacatagaggttgaggatgtcaatgacaatgcaccacagacatcagagcctgtttattacccagaaatcatggaaaattctcctaaagatgtatctgtggtccagatcgaggcatttgatccagattcgagctctaatgacaagctcatgtacaaaattacaagtggaaatccacaaggattcttttcaatacatcctaaaacaggtctcatcacaactacgtcaaggaagctagaccgagaacagcaagatgaacacatattagaggttactgtgacagacaatggtagtccccccaaatcaaccattgcaagagtcattgtgaaaatccttgatgaaaatgacaacaaacctcagtttctgcaaaagttctacaaaatcagactccctgagcgggaaaagccagaccgagaaagaaatgccagacgggagccgctctatcacgtcatagccaccgacaaggatgagggccccaatgcagaaatctcctacagcatcgaagacgggaatgagcatggcaaatttttcatcgaaccgaaaactggagtggtttcgtccaagaggttttcagcagctggagaatatgatattctttcaattaaggcagttgacaatggtcgccctcaaaagtcatcaaccaccagactccatattgaatggatctccaagcccaaaccgtccctggagcccatttcatttgaagaatcattttttacctttactgtgatggaaagtgaccccgttgctcacatgattggagtaatatctgtggagcctcctggcatacccctttggtttgacatcactggtggcaactacgacagtcacttcgatgtggacaagggaactggaaccatcattgttgccaaacctcttgatgcagaacagaagtcaaactacaacctcacagtcgaggctacagatggaaccaccactatcctcactcaggtattcatcaaagtaatagacacaaatgaccatcgtcctcagttttctacatcaaagtatgaagttgttattcctgaagatacagcgccagaaacagaaattttgcaaatcagtgctgtggatcaggatgagaaaaacaaactaatctacactctgcagagcagtagagatccactgagtctcaagaaatttcgtcttgatcctgcaaccggctctctctatacttctgagaaactggatcatgaagctgttcaccagcacaccctcacggtcatggtacgagatcaagatgtgcctgtaaaacgcaactttgcaaggattgtggtcaatgtcagcgacacgaatgaccacgccccgtggttcaccgcttcctcctacaaagggcgggtttatgaatcggcagccgttggctcagttgtgttgcaggtgacggctctggacaaggacaaagggaaaaatgctgaagtgctgtactcgatcgagtcaggaaatattggaaattcttttatgattgatcctgtcttgggctctattaaaactgccaaagaattagatcgaagtaaccaagcggagtatgatttaatggtaaaagctacagataagggcagtccaccaatgagtgaaataacttctgtgcgtatctttgtcacaattgctgacaacgcctctccgaagtttacatcaaaagaatattctgttgaacttagtgaaactgtcagcattgggagtttcgttgggatggttacagcccatagtcaatcatcagtggtgtatgaaataaaagatggaaatacaggtgatgcttttgatattaatccacattctggaactatcatcactcagaaagccctggactttgaaactttgcccatttacacattgataatacaaggaactaacatggctggtttgtccactaatacaacggttctagttcacttgcaggatgagaatgacaacgcgccagtttttatgcaggcagaatatacaggactcattagtgaatcagcctcaattaacagcgtggtcctaacagacaggaatgtcccactggtgattcgagcagctgatgctgataaagactcaaatgctttgcttgtatatcacattgttgaaccatctgtacacacatattttgctattgattctagcactggtgctattcatacagtactaagtctggactatgaagaaacaagtatttttcactttaccgtccaagtgcatgacatgggaaccccacgtttatttgctgagtatgcagcgaatgtaacagtacatgtaattgacattaatgactgcccccctgtgtttgccaagccattatatgaagcatctcttttgttaccaacatacaaaggagtaaaagtcatcacagtaaatgctacagatgctgattcaagtgcattctcacagttgatttactccatcaccgaaggcaacatcggggagaagttttctatggactacaagactggtgctctcactgtccaaaacacaactcagttaagaagccgctacgagctaaccgttagagcttccgatggcagatttgccggccttacctctgtcaaaattaatgtgaaagaaagcaaagaaagtcacctaaagtttacccaggatgtctactctgcggtagtgaaagagaattccaccgaggccgaaacattagctgtcattactgctattgggaatccaatcaatgagcctttgttttatcacatcctcaacccagatcgcagatttaaaataagccgcacttcaggagttctgtcaaccactggcacgcccttcgatcgtgagcagcaggaggcgtttgatgtggttgtagaagtgacagaggaacataagccttctgcagtggcccacgttgtcgtgaaggtcattgtagaagaccaaaatgataatgcgccggtgtttgtcaaccttccctactacgccgttgttaaagtggacactgaggtgggccatgtcattcgctatgtcactgctgtagacagagacagtggcagaaacggggaagtgcattactacctcaaggaacatcatgaacactttcaaattggacccttgggtgaaatttcactgaaaaagcaatttgagcttgacaccttaaataaagaatatcttgttacagtggttgcaaaagatggagggaacccggccttttcagcggaagttatcgttccgatcactgtcatgaataaagccatgcctgtgtttgaaaaacctttctacagtgcagagattgcagagagcatccaggtgcacagccctgtggtccacgtgcaggctaacagcccggaaggcctgaaagtgttctacagcatcacagacggagaccctttcagccagttcactattaacttcaatactggagttatcaatgtcatagctcctctggactttgaggcccacccggcatataagctgagcatacgcgcaactgactccttgacgggcgctcatgctgaagtatttgtggacatcatagtagacgacatcaatgataaccctcctgtgtttgctcagcagtcttatgcggtgaccctgtctgaggcatctgtaattggaacgtctgttgttcaagttagagccaccgattctgattcagaaccaaatagaggaatctcataccagatgtttgggaatcacagcaagagtcatgatcattttcatgtagacagcagcactggcctcatctcactactcagaaccctggattacgagcagtcccggcagcacacgatttttgtgagggcagttgatggtggtatgcccacgctgagcagtgatgtgattgtcacggtggacgttaccgacctcaatgataatccaccactctttgaacaacagatttatgaagccagaattagcgagcacgcccctcatgggcatttcgtgacctgtgtaaaagcctatgatgcagacagttcagacatagacaagttgcagtattccattctgtctggcaatgatcataaacattttgtcattgacagtgcaacagggattatcaccctctcaaacctgcaccggcacgccctgaagccattttacagtcttaacctgtcagtgtctgatggagtttttagaagttccacccaggttcatgtaactgtaattggaggcaatttgcacagtcctgctttccttcagaacgaatatgaagtggaactagctgaaaacgctcccctacataccctggtgatggaggtgaaaactacggatggggattctggtatttatggtcacgttacttaccatattgtaaatgactttgccaaagacagattttacataaatgagagaggacagatatttactttggaaaaacttgatcgagaaaccccggcggagaaagtgatctcagtccgtttaatggctaaggatgctggaggaaaagttgctttctgcaccgtgaatgtcatccttacagatgacaatgacaatgcaccacaatttcgagcaaccaaatacgaagtgaatatcgggtccagtgctgctaaagggacttcagtcgttaaagttcttgcaagtgatgccgatgagggctccaatgccgacatcacctatgccattgaagcagactctgaaagtgtaaaagagaatttggaaattaacaaactgtccggcgtaatcactacaaaggagagcctcattggcttggaaaatgaattcttcactttctttgttagagctgtggataatgggtctccatcaaaagaatctgttgttcttgtctatgttaaaatccttccaccggaaatgcagcttccaaaattttcagaacctttctatacctttacagtgtcagaggacgtgcctattggaacagagatagatctcatccgagcagaacatagtgggactgttctttacagcctggtcaaagggaatactccagaaagcaatagggatgagtcctttgtgattgacagacagagcgggagactgaagttggagaagagtcttgatcatgagacaactaagtggtatcagttttccatactggccaggtgcactcaagatgaccatgagatggtggcttctgtagatgttagtatccaagtgaaagatgcaaatgacaacagcccggtctttgaatctagtccatatgaggcattcattgttgaaaacctgccagggggaagtagagtaattcagatcagggcatctgatgctgactcaggaaccaacggccaagttatgtatagcctggatcagtcacaaagtgtggaagtcattgaatcctttgccattaacatggaaacaggctggattacaactttaaaggaacttgaccatgaaaagagagacaattaccagattaaagtggttgcatcagatcatggtgaaaagatccagctatcctccacagccattgtggatgttaccgtcaccgatgtcaacgatagtccaccacgattcacggccgagatctataaagggactgtgagtgaggatgacccccaaggtggggtgattgccatcttaagtaccacggatgctgattctgaagagatcaacagacaagttacatatttcataacaggaggggatcctttaggacagtttgccgttgaaactatacagaatgaatggaaggtatatgtgaagaaacctctagacagggaaaaaagggacaattaccttcttactatcacggcaactgatggcaccttctcatcaaaagcgatagttgaagtgaaagttctggatgcaaatgacaacagtccagtttgtgaaaagactttatattcagacactattcctgaagacgtccttcctggaaaattgatcatgcagatctctgctacagacgcagacatccgctctaacgctgaaattacttacacgttattgggttcaggtgcagaaaaattcaaactaaatccagacacaggtgaactgaaaacgtcaaccccccttgatcgtgaggagcaagctgtttatcatcttctcgtcagggccacagatggaggaggaagattctgccaagccagtattgtgctcacgctagaagatgtgaacgataacgcccccgaattctctgccgatccttatgccatcaccgtgtttgaaaacacagagccgggaacgctgctgacaagagtgcaggccacagatgccgacgcaggattaaatcggaagattttatactcactgattgactctgctgatgggcagttctccattaacgaattatctggaattattcagttagaaaaacctttggacagagaactccaggcagtatacaccctctctttgaaagctgtggatcaaggcttgccaaggaggctgactgccactggcactgtgattgtatcagttcttgacataaatgacaacccccctgtgtttgagtaccgtgaatatggtgccaccgtgtctgaggacattcttgttggaactgaagttcttcaagtgtatgcagcaagtcgggatattgaagcaaatgcagaaatcacctactcaataataagtggaaatgaacatgggaaattcagcatagattctaaaacaggggccgtatttatcattgagaatctggattatgagagctctcatgagtattacctaacagtagaggccactgatggaggcacgccttcactgagcgacgttgccactgtgaacgttaatgtaacagatatcaacgataatacccctgtgttcagccaagacacctacacgacagtcatcagtgaagatgccgttcttgagcagtctgtcatcacggttatggccgatgatgccgatggaccttccaacagccacatccactactcaattatagatggcaaccaaggaagctcgttcacaattgaccccgtcaggggagaagtcaaagtgaccaaacttctcgaccgagaaacgatttcaggttacacgctcacggttcaagcttctgataatggcagtccacccagagtcaacacgacgaccgtgaacatcgatgtgtccgatgtcaatgacaacgcgcccgtcttctccaggggaaactacagtgtcattatccaggaaaataagccagtgggcttcagcgtgctgcagctggtagtaacagatgaggattcttcccataacggtccacccttcttctttactattgtaactggaaatgatgagaaggcttttgaagttaacccgcaaggagtcctcctgacatcatctgccatcaagaggaaggagaaagatcattacttactgcaggtgaaggtggcagataatggaaagcctcagttgtcatctttgacatacattgacattagggtaattgaggagagcatctatccgcctgcgattttgcccctggagattttcatcacctcttctggagaagaatactcaggtggcgtcattgggaagatccatgccacagaccaggacgtgtatgatactctaacctacagtctcgaccctcagatggacaacctgttctctgtttccagcacagggggcaagctgatagcacacaaaaagctagacatagggcaataccttctcaatgtcagcgtaacagatgggaagttcacgacggtggccgacatcacagtgcatatcagacaagtcacacaggagatgttgaaccacaccatcgcgatccgctttgccaacctcactccggaagaattcgttggtgactactggcgcaacttccagcgagctttacggaacatcctgggtgtgaggaggaacgacatacagattgttagtttgcagtcctctgaacctcacccacatctggacgtcttactttttgtagagaaaccaggtagtgctcagatctcaacaaaacaacttctgcacaagattaactcttccgtgactgacattgaggaaatcattggagttaggatactgaatgtattccagaaactctgcgcgggactggactgcccctggaagttctgcgatgaaaaggtgtctgtggatgaaagtgtgatgtcaacacacagcacagccagactgagttttgtgactccccgccaccacagggcagcggtgtgtctctgcaaagagggaaggtgcccacctgtccaccatggctgtgaagatgatccgtgccctgagggatccgaatgtgtgtctgatccctgggaggagaaacacacctgtgtctgtcccagcggcaggtttggtcagtgcccagggagttcatctatgacactgactggaaacagctacgtgaaataccgtctgacggaaaatgaaaacaaattagagatgaaactgaccatgaggctcagaacatattccacgcatgcggttgtcatgtatgctcgaggaactgactatagcatcttggagattcatcatggaaggctgcagtacaagtttgactgtggaagtggccctggaattgtctctgttcagagcattcaggtcaatgatgggcagtggcacgcagtggccctggaagtgaatggaaactatgctcgcttggttctagaccaagttcatactgcatcgggcacagccccagggactctgaaaaccctgaacctggataactatgtgttttttggtggccacatccgtcagcagggaacaaggcatggaagaagtcctcaagttggtaatggtttcaggggttgtatggactccatttatttgaatgggcaggagctccctttaaacagcaaacccagaagctatgcacacatcgaagagtcggtggatgtatctccaggctgcttcctgacggccacggaagactgcgccagcaacccttgccagaatggaggcgtttgcaatccgtcacctgctggaggttattactgcaaatgcagtgccttgtacatagggacccactgtgagataagcgtcaatccgtgttcctccaagccatgcctctatgggggcacgtgtgttgtcgacaacggaggctttgtttgccagtgtagaggattatatactggtcagaggtgtcagcttagtccatactgcaaagatgaaccctgtaagaatggcggaacatgctttgacagtttggatggcgccgtttgtcagtgtgattcgggttttaggggagaaaggtgtcagagtgatatcgacgagtgctctggaaacccttgcctgcacggggccctctgtgagaacacgcacggctcctatcactgcaactgcagccacgagtacaggggacgtcactgcgaggatgctgcgcccaaccagtatgtgtccacgccgtggaacattgggttggcggaaggaattggaatcgttgtgtttgttgcagggatatttttactggtggtggtgtttgttctctgccgtaagatgattagtcggaaaaagaagcatcaggctgaacctaaagacaagcacctgggacccgctacggctttcttgcaaagaccgtattttgattccaagctaaataagaacatttactcagacataccaccccaggtgcctgtccggcctatttcctacaccccgagtattccaagtgactcaagaaacaatctggaccgaaattccttcgaaggatctgctatcccagagcatcccgaattcagcacttttaaccccgagtctgtgcacgggcaccgaaaagcagtggcggtctgcagcgtggcgccaaacctgcctcccccacccccttcaaactccccttctgacagcgactccatccagaagcctagctgggactttgactatgacacaaaagtggtggatcttgatccctgtctttccaagaagcctctagaggaaaagccttcccagccatacagtgcccgggaaagcctgtctgaagtgcagtctctgagctccttccagtccgaatcgtgcgatgacaatgggtatcactgggatacatcagattggatgccaagcgttcctctgccggacatacaagagttccccaactatgaggtgattgatgagcagacacccctgtactcagcagatccaaacgccatcgatacggactattaccctggaggctacgacatcgaaagtgattttcctccacccccagaagacttccccgcagctgatgagctaccaccgttaccgcccgaattcagcaatcagtttgaatccatccaccctcctagagacatgcctgccgcgggtagcttgggttcttcatcaagaaaccggcagaggttcaacttgaatcagtatttgcccaatttttatcccctcgatatgtctgaacctcaaacaaaaggcactggtgagaatagtacttgtagagaaccccatgccccttacccgccagggtatcaaagacacttcgaggcgcccgctgtcgagagcatgcccatgtctgtgtacgcctccaccgcctcctgctctgacgtgtcagcctgctgcgaagtggagtccgaggtcatgatgagtgactatgagagcggggacgacggccacttcgaagaggtgacgatcccgcccctggattcccagcagcacacggaagtctgactctcaactccccccaaagtgcctgactttagtgaacctagaggtgatgtgagtaatccgcgctgttctttgcagcagtgcttccaagctttttttggtgagccgaatgggcatggctgcgctggatcctgcgcctctggacgtgctagccatttccagtgtcccaactactgtcatcgtgaggttttcatcggctgtgccatttcccaacgtcttttgggatttacatctgtctgtgttaaaataatcaaacgaaaaatcagtcctgtgttgtcagcatgattcatgtatttatatagatttgattattttaattttcctgtctcttttttttgtaaattttatgtacagatttgatttttcatagttttaactagatttccaagatattttgtgcatttgtttcaactgaattttggtggtgtcagtgccattatctagcaccctgatttttttttttttactataaccagggtttcattctgtctttttccactgaagtgtgacattttgttagtacatttcagtgtagtcattcatttctagctgtacataggatgaaggagagatcagatacatgaacatgtcttacatgggttgctgtatttagaattataaacatttttcattattggaaagtgtaacggggaccttctgcatacctgtttagaaccaaaaccaccatgacacagtttttatagtgtctgtatatttgtgatgcaatggtcttgtaaaggtttttaatgaaaactaccattagccagtctttcttactgacaataaattattaataaaatacttgagctttaaaaaaaaaa

SEQ ID NO: 2 - protein, 4588 aa, Homo sapiens
MGRHLALLLLLLLLFQHFGDSDGSQRLEQTPLQFTHLEYNVTVQENSAAKTYVGHPVKMGVYITHPAWEVRYKIVSGDSENLFKAEEYILGDFCFLRIRTKGGNTAILNREVKDHYTLIVKALEKNTNVEARTKVRVQVLDTNDLRPLFSPTSYSVSLPENTAIRTSIARVSATDADIGTNGEFYYSFKDRTDMFAIHPTSGVIVLTGRLDYLETKLYEMEILAADRGMKLYGSSGISSMAKLTVHIEQANECAPVITAVTLSPSELDRDPAYAIVTVDDCDQGANGDIASLSIVAGDLLQQFRTVRSFPGSKEYKVKAIGGIDWDSHPFGYNLTLQAKDKGTPPQFSSVKVIHVTSPQFKAGPVKFEKDVYRAEISEFAPPNTPVVMVKAIPAYSHLRYVFKSTPGKAKFSLNYNTGLISILEPVKRQQAAHFELEVTTSDRKASTKVLVKVLGANSNPPEFTQTAYKAAFDENVPIGTTVMSLSAVDPDEGENGYVTYSIANLNHVPFAIDHFTGAVSTSENLDYELMPRVYTLRIRASDWGLPYRREVEVLATITLNNLNDNTPLFEKINCEGTIPRDLGVGEQITTVSAIDADELQLVQYQIEAGNELDFFSLNPNSGVLSLKRSLMDGLGAKVSFHSLRITATDGENFATPLYINITVAASHKLVNLQCEETGVAKMLAEKLLQANKLHNQGEVEDIFFDSHSVNAHIPQFRSTLPTGIQVKENQPVGSSVIFMNSTDLDTGFNGKLVYAVSGGNEDSCFMIDMETGMLKILSPLDRETTDKYTLNITVYDLGIPQKAAWRLLHVVVVDANDNPPEFLQESYFVEVSEDKEVHSEIIQVEATDKDLGPNGHVTYSIVTDTDTFSIDSVTGVVNIARPLDRELQHEHSLKIEARDQAREEPQLFSTVVVKVSLEDVNDNPPTFIPPNYRVKVREDLPEGTVIMWLEAHDPDLGQSGQVRYSLLDHGEGNFDVDKLSGAVRIVQQLDFEKKQVYNLTVRAKDKGKPVSLSSTCYVEVEVVDVNENLHPPVFSSFVEKGTVKEDAPVGSLVMTVSAHDEDARRDGEIRYSIRDGSGVGVFKIGEETGVIETSDRLDRESTSHYWLTVFATDQGVVPLSSFIEIYIEVEDVNDNAPQTSEPVYYPEIMENSPKDVSVVQIEAFDPDSSSNDKLMYKITSGNPQGFFSIHPKTGLITTTSRKLDREQQDEHILEVTVTDNGSPPKSTIARVIVKILDENDNKPQFLQKFYKIRLPEREKPDRERNARREPLYHVIATDKDEGPNAEISYSIEDGNEHGKFFIEPKTGVVSSKRFSAAGEYDILSIKAVDNGRPQKSSTTRLHIEWISKPKPSLEPISFEESFFTFTVMESDPVAHMIGVISVEPPGIPLWFDITGGNYDSHFDVDKGTGTIIVAKPLDAEQKSNYNLTVEATDGTTTILTQVFIKVIDTNDHRPQFSTSKYEVVIPEDTAPETEILQISAVDQDEKNKLIYTLQSSRDPLSLKKFRLDPATGSLYTSEKLDHEAVHQHTLTVMVRDQDVPVKRNFARIVVNVSDTNDHAPWFTASSYKGRVYESAAVGSVVLQVTALDKDKGKNAEVLYSIESGNIGNSFMIDPVLGSIKTAKELDRSNQAEYDLMVKATDKGSPPMSEITSVRIFVTIADNASPKFTSKEYSVELSETVSIGSFVGMVTAHSQSSVVYEIKDGNTGDAFDINPHSGTIITQKALDFETLPIYTLIIQGTNMAGLSTNTTVLVHLQDENDNAPVFMQAEYTGLISESASINSVVLTDRNVPLVIRAADADKDSNALLVYHIVEPSVHTYFAIDSSTGAIHTVLSLDYEETSIFHFTVQVHDMGTPRLFAEYAANVTVHVIDINDCPPVFAKPLYEASLLLPTYKGVKVITVNATDADSSAFSQLIYSITEGNIGEKFSMDYKTGALTVQNTTQLRSRYELTVRASDGRFAGLTSVKINVKESKESHLKFTQDVYSAVVKENSTEAETLAVITAIGNPINEPLFYHILNPDRRFKISRTSGVLSTTGTPFDREQQEAFDVVVEVTEEHKPSAVAHVVVKVIVEDQNDNAPVFVNLPYYAVVKVDTEVGHVIRYVTAVDRDSGRNGEVHYYLKEHHEHFQIGPLGEISLKKQFELDTLNKEYLVTVVAKDGGNPAFSAEVIVPITVMNKAMPVFEKPFYSAEIAESIQVHSPVVHVQANSPEGLKVFYSITDGDPFSQFTINFNTGVINVIAPLDFEAHPAYKLSIRATDSLTGAHAEVFVDIIVDDINDNPPVFAQQSYAVTLSEASVIGTSVVQVRATDSDSEPNRGISYQMFGNHSKSHDHFHVDSSTGLISLLRTLDYEQSRQHTIFVRAVDGGMPTLSSDVIVTVDVTDLNDNPPLFEQQIYEARISEHAPHGHFVTCVKAYDADSSDIDKLQYSILSGNDHKHFVIDSATGIITLSNLHRHALKPFYSLNLSVSDGVFRSSTQVHVTVIGGNLHSPAFLQNEYEVELAENAPLHTLVMEVKTTDGDSGIYGHVTYHIVNDFAKDRFYINERGQIFTLEKLDRETPAEKVISVRLMAKDAGGKVAFCTVNVILTDDNDNAPQFRATKYEVNIGSSAAKGTSVVKVLASDADEGSNADITYAIEADSESVKENLEINKLSGVITTKESLIGLENEFFTFFVRAVDNGSPSKESVVLVYVKILPPEMQLPKFSEPFYTFTVSEDVPIGTEIDLIRAEHSGTVLYSLVKGNTPESNRDESFVIDRQSGRLKLEKSLDHETTKWYQFSILARCTQDDHEMVASVDVSIQVKDANDNSPVFESSPYEAFIVENLPGGSRVIQIRASDADSGTNGQVMYSLDQSQSVEVIESFAINMETGWITTLKELDHEKRDNYQIKVVASDHGEKIQLSSTAIVDVTVTDVNDSPPRFTAEIYKGTVSEDDPQGGVIAILSTTDADSEEINRQVTYFITGGDPLGQFAVETIQNEWKVYVKKPLDREKRDNYLLTITATDGTFSSKAIVEVKVLDANDNSPVCEKTLYSDTIPEDVLPGKLIMQISATDADIRSNAEITYTLLGSGAEKFKLNPDTGELKTSTPLDREEQAVYHLLVRATDGGGRFCQASIVLTLEDVNDNAPEFSADPYAITVFENTEPGTLLTRVQATDADAGLNRKILYSLIDSADGQFSINELSGIIQLEKPLDRELQAVYTLSLKAVDQGLPRRLTATGTVIVSVLDINDNPPVFEYREYGATVSEDILVGTEVLQVYAASRDIEANAEITYSIISGNEHGKFSIDSKTGAVFIIENLDYESSHEYYLTVEATDGGTPSLSDVATVNVNVTDINDNTPVFSQDTYTTVISEDAVLEQSVITVMADDADGPSNSHIHYSIIDGNQGSSFTIDPVRGEVKVTKLLDRETISGYTLTVQASDNGSPPRVNTTTVNIDVSDVNDNAPVFSRGNYSVIIQENKPVGFSVLQLVVTDEDSSHNGPPFFFTIVTGNDEKAFEVNPQGVLLTSSAIKRKEKDHYLLQVKVADNGKPQLSSLTYIDIRVIEESIYPPAILPLEIFITSSGEEYSGGVIGKIHATDQDVYDTLTYSLDPQMDNLFSVSSTGGKLIAHKKLDIGQYLLNVSVTDGKFTTVADITVHIRQVTQEMLNHTIAIRFANLTPEEFVGDYWRNFQRALRNILGVRRNDIQIVSLQSSEPHPHLDVLLFVEKPGSAQISTKQLLHKINSSVTDIEEIIGVRILNVFQKLCAGLDCPWKFCDEKVSVDESVMSTHSTARLSFVTPRHHRAAVCLCKEGRCPPVHHGCEDDPCPEGSECVSDPWEEKHTCVCPSGRFGQCPGSSSMTLTGNSYVKYRLTENENKLEMKLTMRLRTYSTHAVVMYARGTDYSILEIHHGRLQYKFDCGSGPGIVSVQSIQVNDGQWHAVALEVNGNYARLVLDQVHTASGTAPGTLKTLNLDNYVFFGGHIRQQGTRHGRSPQVGNGFRGCMDSIYLNGQELPLNSKPRSYAHIEESVDVSPGCFLTATEDCASNPCQNGGVCNPSPAGGYYCKCSALYIGTHCEISVNPCSSKPCLYGGTCVVDNGGFVCQCRGLYTGQRCQLSPYCKDEPCKNGGTCFDSLDGAVCQCDSGFRGERCQSDIDECSGNPCLHGALCENTHGSYHCNCSHEYRGRHCEDAAPNQYVSTPWNIGLAEGIGIVVFVAGIFLLVVVFVLCRKMISRKKKHQAEPKDKHLGPATAFLQRPYFDSKLNKNIYSDIPPQVPVRPISYTPSIPSDSRNNLDRNSFEGSAIPEHPEFSTFNPESVHGHRKAVAVCSVAPNLPPPPPSNSPSDSDSIQKPSWDFDYDTKVVDLDPCLSKKPLEEKPSQPYSARESLSEVQSLSSFQSESCDDNGYHWDTSDWMPSVPLPDIQEFPNYEVIDEQTPLYSADPNAIDTDYYPGGYDIESDFPPPPEDFPAADELPPLPPEFSNQFESIHPPRDMPAAGSLGSSSRNRQRFNLNQYLPNFYPLDMSEPQTKGTGENSTCREPHAPYPPGYQRHFEAPAVESMPMSVYASTASCSDVSACCEVESEVMMSDYESGDDGHFEEVTIPPLDSQQHTEV

SEQ ID NO: 3 - DNA, 20 bp, Homo sapiens
gtgtgattcgggttttaggg

SEQ ID NO: 4 - DNA, 20 bp, Homo sapiens
ctgtactcgtggctgcagtt

SEQ ID NO: 5 - DNA, 19 bp, Homo sapiens
gtctactgctggtcttggc

SEQ ID NO: 6 - DNA, 20 bp, Homo sapiens
ctctggtggcttgcaacatc

SEQ ID NO: 7 - DNA, 26 bp, Homo sapiens
ctgttgatgtctttctgtgtagctac

SEQ ID NO: 8 - DNA, 24 bp, Homo sapiens
cattcgacgatagtacttagccag

SEQ ID NO: 9 - DNA, 21 bp, Homo sapiens
gccaatgaaaccaggtatccc

SEQ ID NO: 10 - DNA, 24 bp, Homo sapiens
gctcaagtaaacaggctgttttcc

SEQ ID NO: 11 - DNA, 31 bp, Homo sapiens
tggagataacactctaagcataactaaaggt

SEQ ID NO: 12 - DNA, 21 bp, Homo sapiens
gatgtagttgcttgggaccca

SEQ ID NO: 13 - DNA, 20 bp, Homo sapiens
ggcatcctcaccctgaagta

SEQ ID NO: 14 - DNA, 20 bp, Homo sapiens
ccatctcttgctcgaagtcc

(19) United States
(12) Patent Application Publication
(10) Pub. No.: US 2013/0129734 A1
(43) Pub. Date: May 23, 2013

(54) COMPOSITION AND METHODS FOR THE DIAGNOSIS, PROGNOSIS AND TREATMENT OF LEUKEMIA

(71) Applicant: Newcastle Innovation Limited, Callaghan (AU)

(72) Inventors: RICK FRANCIS THORNE, NEW LAMBTON (AU); CHARLES EDO DE BOCK, ISLINGTON (AU); GORDON FROOD BURNS, NEW LAMBTON HEIGHTS (AU)

(73) Assignee: NEWCASTLE INNOVATION LIMITED, CALLAGHAN (AU)

(21) Appl. No.: 13/675,978

(22) Filed: Nov. 13, 2012

Related U.S. Application Data
(60) Provisional application No. 61/559,639, filed on Nov. 14, 2011.

(51) Int. Cl. A61K 39/00; C12Q 1/68; G01N 33/68; A61K 39/395; A61K 45/06

(52) U.S. Cl. CPC A61K 39/0011; A61K 39/39558; A61K 45/06; G01N 33/6893; C12Q 1/6886
USPC 424/136.1; 424/138.1; 424/133.1; 424/178.1; 435/7.23; 435/6.11; 424/277.1

(57) ABSTRACT

The present disclosure relates generally to compositions and methods for the diagnosis, prognosis and treatment of leukemia, in particular leukemia in which leukemic cells, or neoplastic precursors thereof, express Fat1 or a homolog of Fat1 that is substantially not expressed on normal blood cells.

### FIELD

The present disclosure relates generally to compositions and methods for the diagnosis, prognosis and treatment of leukemia.

### BACKGROUND

Leukemia is a significant and debilitating form of cancer affecting blood and bone marrow cells. Most leukemias begin with a malignant transformation of haematopoietic precursors in the bone marrow, which leads to overcrowding of the bone marrow and a reduction in its capacity to make normal blood cells. Increasing numbers of abnormal blast cells (or leukemic blast cells) eventually spill out into the circulation, which explains why leukemia is often characterised by an increase in the number of circulating white blood cells.

It is a disease that is associated with significant morbidity and mortality. In 2007 in Australia, it was estimated that around 3,000 people, including 250 children (0-14 years), would be diagnosed with leukemia. More recently, the National Cancer Institute estimated that, in 2011 in the United States, there would be 44,600 new cases and 21,780 deaths arising from leukemia.

Many patients who go into remission following initial treatment are at risk of relapse. For example, children with acute lymphoblastic leukemia (ALL) continue to suffer a 20% incidence of relapse after treatment with the best available therapy. High-resolution genomic profiling, including analysis of single-nucleotide polymorphisms and copy number abnormalities, has greatly aided an understanding of the molecular mechanisms underlying treatment outcome, therapy response and the biology of relapse.

Genomic studies have shown that copy number abnormalities in genes involved in lymphoid differentiation and cell cycle control are common. For precursor B-cell (preB) ALL, for example, deletions, or part thereof, are found in PAX5, EBF1, IKZF1, TCF-4, CDKN2A and RB1. Recent reports also indicate that deletions and nonsense mutations of the HUH gene are significantly associated with poor relapse-free and overall survival rates in preB-ALL. However, in light of these studies, questions remain on the biology of relapse, with marker analysis complicated by the fact that phenotypic shifts in preB-ALL blasts can occur between diagnostic and post-chemotherapy or relapse samples. Those cells that give rise to relapse in some cases appear to be selected during treatment, with clonal evolution occurring of a minor subclone present at diagnosis rather than simply being the development of chemotherapeutic resistance of the original leukemic clone. The inherent genetic heterogeneity has more recently been described within subpopulations of leukemia-initiating cells, which also undergo dynamic and branching evolution. This evolution leads to shifts in subclone dominance during progression and treatment relapse, further highlighting the clinical challenge in delivering targeted therapies against differential markers expressed by the majority of clones if minor subclones then survive and undergo further evolution, leading to relapse.

Overall, the use of genomic profiling technology has been very informative on identifying novel genetic alterations in leukemia, but it has been noted that a proportion of cases of leukemia, such as ALL, with no discernable cytogenetic changes also fail therapy (12). Hence, there is a need to identify highly selective markers for the diagnosis, prognosis and treatment of leukemia.

### SUMMARY

Aspects disclosed herein are based on the surprising findings that leukemic cells can be distinguished from non-leukemic cells by virtue of the differential expression of Fat1 cadherin on leukemic cells.

Accordingly, an aspect enabled herein is a method for treating a subject with a leukemia, the method comprising administering to the subject an effective amount of an agent that is selectively cytotoxic to leukemic cells, or neoplastic precursors thereof, that express Fat1 or a homolog of Fat1 that is substantially not expressed on normal blood cells.

Another aspect enabled herein is use of an agent that is selectively cytotoxic to leukemic cells or neoplastic precursors thereof that express Fat1, or a homolog of Fat1 that is substantially not expressed on normal blood cells, in the manufacture of a medicament for the treatment of a subject with leukemia.

Another aspect enabled herein is a composition comprising an agent that is selectively cytotoxic to leukemic cells or neoplastic precursors thereof that express Fat1, or a homolog of Fat1 that is substantially not expressed on normal blood cells, and one or more pharmaceutically acceptable carriers, diluents or excipients.

Another aspect enabled herein is a method for the diagnosis or prognosis of a leukemia in a subject, the method comprising executing the step of analyzing a blood sample from the subject for the presence of cells that express Fat1 or a homolog thereof that is substantially not expressed on normal blood cells, wherein the execution step comprises contacting a primary binding agent that specifically binds to Fat1 on blood cells, wherein the binding of the primary binding agent to the cells is indicative of presence of cells that express Fat1 or its homolog and provides an indication of the presence of leukemic cells or precursors thereof.

Another aspect enabled herein is a method for the diagnosis or prognosis of a leukemia in a subject, the method comprising executing the step of analyzing a blood sample from the subject for the presence of cells that express Fat1 or a homolog thereof that is substantially not expressed by normal blood cells, wherein the execution step comprises contacting nucleic acid from the blood sample with an oligonucleotide probe that is capable of hybridizing to a nucleic acid sequence encoding Fat1, or a homolog thereof, wherein the binding of the probe to the nucleic acid from the blood sample is indicative of presence of a cell that expresses Fat1 or its homolog and provides an indication of the presence of a leukemic cell or a precursor thereof.

Another aspect enabled herein is a therapeutic protocol for treating leukemia in a subject, said protocol comprising the steps of:
a. executing the step of analyzing a sample of blood from the subject for the presence of cells that express Fat1 or a homolog thereof that is substantially not expressed on normal blood cells, wherein the execution step comprises contacting a primary binding agent that specifically binds to Fat1 on blood cells, wherein the presence of cells that express Fat1 is indicative of the presence of leukemic cells or a precursor form thereof;
b. administering to a subject who contains Fat1-expressing cells an agent that is selectively cytotoxic to leukemic cells, or neoplastic precursors thereof that express Fat1 or a homolog of Fat1 that is substantially not expressed on normal blood cells;
c. monitoring for a reduction in the presence of Fat1-expressing cells over time;
   wherein a reduction in Fat1-expressing cells over a period of time is indicative of a successful treatment.

Another aspect enabled herein is a therapeutic protocol for treating leukemia in a subject, said protocol comprising the steps of:
a. executing the step of analyzing a sample of blood from the subject for the presence of cells that express Fat1 or a homolog thereof that is substantially not expressed on normal blood cells, wherein the execution step comprises contacting nucleic acid from the blood sample with a probe that is capable of hybridizing under stringent conditions to a nucleic acid sequence encoding Fat1, or a homolog thereof, wherein the binding of the probe to the nucleic acid from the blood sample is indicative of presence of a cell that expresses Fat1 or its homolog and provides an indication of the presence of a leukemic cell or a precursor thereof;
b. administering to a subject who contains Fat1-expressing cells an agent that is selectively cytotoxic to leukemic cells, or neoplastic precursors thereof that express Fat1 or a homolog of Fat1 that is substantially not expressed on normal blood cells; and
c. monitoring for a reduction in the presence of Fat1-expressing cells over time;
   wherein a reduction in Fat1-expressing cells over a period of time is indicative of a successful treatment.

Another aspect enabled herein is a method of vaccinating a subject against leukemia, the method comprising administering to the subject an amount of a compound comprising a Fat1 polypeptide, or a immunogenic fragment thereof, effective to stimulate antibodies against Fat1 expressed by cells in the subject.

### BRIEF DESCRIPTION OF THE FIGURES

FIG. 1 shows Fat1 cadherin protein expression in leukemia cell line panel. (a) Schematic diagram of the full-length Fat1 cadherin protein, which is 4588 amino acids long and has a predicted molecular weight of 550 kDa. It is a type I transmembrane protein with 34 extracellular cadherin domains, 5 epidermal growth factor-like motifs and 1 laminin G-like domain. (b) The expression of Fat1 protein in a leukemia cell line panel reveals an immunoreactive band at the predicted molecular weight for Fat1 at 550 kDa in three of the four T-cell ALLs (Jurkat, JM and MOLT-4), both preB-ALLs (Nalm-6 and LK63) and one of the two AMLs (THP-1) examined. There was no visible expression of Fat1 in the acute promyelocytic leukemia (HL60), erythroleukemia (HEL), B-cell ALL (Balm-1) or lymphoma (RAH and KARPAS) cell lines. Similarly, normal peripheral blood (PB) cells from four separate healthy donors had no detectable Fat1 protein expression. (c) qPCR analysis for Fat1 mRNA of the same cell lines and normal PB cells from healthy donors generally reconciled with protein levels, except for both HL-60 and HPB-ALL, which had significant Fat1 mRNA signal but no equivalent full-length Fat1 protein present as measured by Western blot.

FIG. 2a shows Fat1 cadherin protein expression is negligible in HSCs isolated from PB or bone marrow (BM). (i) Illustration of the simplified gating strategy used to fluorescence-activated cell sort circulating hematopoietic stem cells (HSCs) from PB. After defining CD34 positive HSCs by ISHAGE gating, the small CD34 positive population (0.1%) was divided into CD133dim and CD133high subpopulations, with sorted gates indicated in green and red, respectively. (ii) Post-sort analysis to validate the purity of each population.

FIGS. 2b through 2d show Fat1 cadherin gene expression is negligible in HSCs isolated from PB or BM. (b) Total RNA from the sorted PB populations was isolated, and the expression of Fat1, CD34 and CD133 measured by qPCR. The relative mRNA expression levels were calculated after normalizing against the GusB and ABL housekeeping genes, resulting in no detectable Fat1 transcript. (c) qPCR analysis of Fat1, CD34 and CD133 mRNA expression in CD34 positive and CD133 positive cells enriched from BM using magnetic-bead-based sorting, showing that Fat1 transcript is lower in enriched progenitors compared with presorted population. All results are representative of at least two independent experiments, (d) in silico analysis of Fat1 mRNA expression in HSC1 (CD133⁺/CD34^{dim}) and HSC2 (CD38⁻, CD34⁺) hematopoietic progenitor populations shows no significant Fat1 expression.

FIG. 3 shows Fat1 mRNA expression tracked during differentiation of the major hematopoietic lineages. Analysis of the data set GSE24759 (2) shows Fat1 mRNA expression from early hematopoietic progenitors (HSC1-CD133⁻/CD34^{dim} and HSC2-CD38⁻/CD34⁺), intermediate populations and differentiated cells across six different lineage signatures relative to differentiation markers used in the fluorescence-activated cell sorting. In each lineage signature, the expression of the primitive marker CD34 decreased concordant with increases in positive expression of lineage-specific differentiation markers. The only significant (P<0.05) level of Fat1 mRNA expression occurs during the final stages of erythropoiesis.

FIG. 4 shows Fat1 is expressed in clinically relevant BM samples of leukemia. qPCR analysis of preB-ALL, T-ALL and AML in concert with cell lines Jurkat, LK63, Nalm-6 and Raji were analyzed for Fat1 expression relative to β-actin and then normalized to LK63 (set to 1). The level of Fat1 mRNA signal is varied, and using the raw Fat1 qPCR signal associated with Nalm-6 (2DCt 40.06; Table 7, Table 8), there are 10/18 Fat1 positive for B-ALL, 18/19 Fat1 positive for T-ALL and Fat1 1/7 positive for AML.

FIG. 5 shows Fat1 expression is associated with poor prognosis in paired diagnosis-relapse samples of preB ALL. (a) Kaplan-Meier plot of relapse-free survival in 32 patients with preB-ALL from GEO data set GSE3912. Those patients expressing high Fat1 (solid line, upper quartile) versus medium/low levels of Fat1 (dotted line, remaining 75%) have significantly poorer outcome (hazard ratio (HR)=5.1, P=0.002). (b) Kaplan-Meier plots for 27 preB-ALL patients in GEO data set GSE18497. Patients with high Fat1 (solid line, upper quartile) had a significantly higher incidence of relapse compared with those expressing lower Fat1 (dotted line, remaining 75%; HR=3.0, P=0.008). (c) Similarly, patients with high Fat1 in GSE18497 (solid line, upper quartile) had a significantly poorer overall survival compared with those expressing lower Fat1 (dotted line, remaining 75%; HR=2.9, P=0.006).

FIG. 6 shows an Oncomine bar chart and equivalent box plot for Fat1 signal intensity in relation to cytogenetics in B-ALL and T-ALL of the Ross leukemia data set (20). Lower panel box plot values represent maximum, 90^{th} percentile, 75^{th} percentile, median, 25^{th} percentile, 10^{th} percentile, minimum. Median Fat expression is highest in those samples carrying the E2A-PBX1 translocation. Graphs were generated in Oncomine (available on the world-wide web at: oncomine.org).

FIG. 7 shows an Oncomine bar chart and equivalent box plot for Fat1 signal intensity in relation to cytogenetics in B-ALL and T-ALL of the Yeoh leukemia data set (21). Lower panel box plot values represent maximum, 90^{th} percentile, 75^{th} percentile, median, 25^{th} percentile, 10^{th} percentile, minimum. Median Fat expression is highest in those samples carrying the E2A-PBX1 translocation. Graphs were generated in Oncomine (available on the world-wide web at: oncomine.org).

### DETAILED DESCRIPTION

Throughout this specification, unless the context requires otherwise, the word “comprise”, or variations such as “comprises” or “comprising”, will be understood to imply the inclusion of a stated element or integer or method step or group of elements or integers or method steps but not the exclusion of any other element or integer or method step or group of elements or integers or method steps.

Nucleotide and amino acid sequences are referred to by a sequence identifier number (SEQ ID NO). The SEQ ID NOs correspond numerically to the sequence identifiers <400>1 (SEQ ID NO:1), <400>2 (SEQ ID NO:2), etc. A sequence listing is provided after the claims.

As used in the subject specification, the singular forms “a”, “an” and “the” include plural aspects unless the context clearly dictates otherwise. Thus, for example, reference to “a leukemia” includes a single leukemia, as well as two or more leukemias; reference to “an agent” includes a single agent, as well as two or more agents; reference to “the disclosure” includes a single and multiple aspects described in the disclosure; and so forth. All aspects disclosed, described and/or claimed herein are encompassed by the term “invention”. Such aspects are enabled across the width of the present invention.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

Aspects disclosed herein are based on the surprising findings that leukemic cells can be distinguished from non-leukemic cells by virtue of the differential expression of Fat1 cadherin on leukemic cells as compared to non-leukemic cells.

Accordingly, in an aspect of the present disclosure, there is provided a method for treating a subject with a leukemia, said method comprising administering to said subject an effective amount of an agent that is selectively cytotoxic to leukemic cells, or neoplastic precursors thereof, that express Fat1 or a homolog of Fat1 that is substantially not expressed on normal blood cells.

The terms “Fat tumour suppressor homolog 1”, “Fat1” and “Fat1 cadherin” are used interchangeably herein and denote the type I transmembrane protein encoded by a gene product that is a member of the cadherin superfamily, a group of integral membrane proteins characterized by the presence of cadherin-type repeats. The Fat1 gene was first cloned in Drosophila and was found to encode a tumour suppressor essential for controlling cell proliferation during Drosophila development. The human homolog of Fat1 is encoded by the nucleic acid sequence SEQ ID NO:1 (Genbank Accession No. NM_{—}005245). The amino acid sequence of human Fat1 is shown as SEQ ID NO:2 (Genbank Accession No. NP_{—}005236.2).

Human Fat1 was cloned from a T-leukemia cell line and shown to encode a type I transmembrane protein with 34 extracellular cadherin repeats, and named after an orthologous drosophila gene called fat that functions as a tumour suppressor. In situ hybridization has shown that Fat1 mRNA expression is present in some epithelial and mesenchymal compartments, but high expression is found only in fetal as opposed to adult tissues. Subsequent cloning of the Fat1 gene in the rat, mouse and zebrafish showed that this molecule is highly conserved in vertebrates and confirms that its expression is developmentally regulated and largely restricted to fetal tissues. A number of studies have analyzed Fat1 expression in cancer, with loss of membranous Fat1 expression correlated with more aggressive tumours for intrahepatic cholangiocarcinoma (Settakorn et al., 2005). In silico analysis of Fat1 expression has shown expression in gastric, pancreatic, colorectal, breast, lung and brain cancers (Katoh et al., 2006).

The terms “homolog” and “isoforms” are used interchangeably herein and their meaning would be understood by those skilled in the art. Examples of a Fat1 homologs and isoforms include gene or protein sequences that share structural and functional similarity to human Fat1 (nucleotide sequence set forth in SEQ ID NO:1 and corresponding amino acid sequence set forth in SEQ ID NO:2), including gene and protein sequences from non-human animals. The terms “homolog” and “isoforms” include both orthologs, which are sequences in different species that are structurally similar due to evolution from a common ancestor, and paralogs, which are similar sequences within the same genome. The term “Fat1” shall be taken to also include Fat1 homologs, unless otherwise stated.

Fat1 homologs also include variants and fragments of the Fat1 protein or nucleic acid sequences encoding such variants and/or fragments. Examples of Fat1 isoforms include those that may result from alternative transcription initiation codons downstream from the 5′ region of the Fat1 genomic sequence (e.g., as located on chromosome 4 of the human genome), leading to alternative exon usage and/or retained intron regions. It would be understood by those skilled in the art that the Fat1 homologs and isoforms encompassed by the present disclosure will be differentially expressed by leukemic cells as compared to normal blood cells.

The term “substantially”, as used herein for purposes of the present disclosure, refers to the expression of Fat1 (or a homolog thereof) as being almost totally or completely absent from the surface of the normal blood cell. For example, the level of expression of Fat1 on a normal blood cell may be 10% or less than the level of expression typically seen on a leukemic cell, wherein the difference in the level of expression is such that a skilled addressee can differentiate between leukemic cells (or neoplastic precursors thereof) and normal blood cells.

The terms “leukemic cell” or “leukemic cells”, as used herein for purposes of the present disclosure, refer to one or more cells or cell types of mammalian origin (e.g., of human origin) having a phenotype and genotype typical of those found in patients with acute or chronic leukemia (e.g., acute myeloid leukemia, chronic myelomonocytic leukemia, acute lymphoblastic leukemia and plasma cell leukemia). Examples of leukemic cells include, but are not limited to, myeloid and lymphocytic cells derived from patients with acute myeloid leukemia (AML), acute lymphocytic leukemia (ALL), chronic myeloid leukemia (CML), chronic lymphocytic leukemia (CLL), erythroleukemia, thrombocythemia and myelodysplastic syndromes. In an embodiment of the disclosure enabled herein, the leukemia cell is an acute lymphoblastic leukemia cell. In an embodiment, the leukemic cell is a B- or T-lineage cell.

Leukemia is also a disease that affects non-human animals. For instance, most forms of leukemia reported in humans having been reported in animals such as horses, pigs, cats, cattle, mice, chickens and a variety of wild animals. Accordingly, a leukemic cell, as used herein, is not limited to a human leukemic cell, but also includes a leukemic cell found in non-human animals.

Neoplastic precursors that give rise to leukemic cells would be known to those skilled in the art. Examples include neoplastic multipotent haematopoietic stem cells.

The term “subject” as used herein refers to an animal which includes a primate, a lower or higher primate. A higher primate includes human. However, it would be understood that both human and non-human animals may benefit from the composition and methods as herein disclosed. A subject regardless of whether a human or non-human animal may be referred to as an individual, subject, animal, patient, host or recipient. Aspects disclosed herein have both human and veterinary applications. For convenience, an “animal” includes livestock and companion animals such as cattle, horses, sheep, pigs, camelids, goats, donkeys, dogs and cats. With respect to horses, these include horses used in the racing industry as well as those used recreationally or in the livestock industry. Examples of laboratory test animals include mice, rats, rabbits, guinea pigs and hamsters. Rabbits and rodent animals, such as rats and mice, provide a convenient test system or animal model as do primates and lower primates.

The subject being treated may present at various stages of disease progression and may have been previously treated for leukemia or other cancer. In an embodiment disclosed herein, the subject is in remission. Despite being in remission, patients are still at risk of relapse and may therefore benefit from the method of treatment enabled by the present disclosure.

The terms “agent”, “chemical agent”, “pharmacologically active agent”, “medicament”, “active” and “drug” are used interchangeably herein to refer to a compound that selectively targets leukemic cells, or neoplastic precursors thereof, that express Fat1 or a homolog of Fat1 that is substantially not expressed on normal blood cells. The desired effect of targeting Fat1-expressing leukemic cells is cell death. Cell death may be initiated, for example, via complement-dependent, antibody-mediated lysis or apoptotic cell death. The terms “agent”, “chemical agent”, “pharmacologically active agent”, “medicament”, “active” and “drug” also encompass pharmaceutically acceptable and pharmacologically active ingredients of those agents mentioned herein, including, but not limited to, salts, esters, amides, prodrugs, active metabolites, analogs, mimetics functional equivalents and the like. When the terms “compound”, “agent”, “chemical agent” “pharmacologically active agent”, “medicament”, “active”, “drug” and “antagonist” are used, it is to be understood that this includes the active agent per se as well as pharmaceutically acceptable, pharmacologically active salt, ester, amide, prodrug, metabolite and analogs thereof.

“Treatment” or “treating” leukemia includes, but is not limited to (i) preventing the proliferation of leukemic cells, or neoplastic precursors thereof, and (ii) diminishing or eliminating leukemic cells, or their neoplastic precursors, in the subject.

The terms “effective amount” or “pharmaceutically effective amount” of a composition or agent, as provided herein, refer to a nontoxic but sufficient amount of the agent to provide a positive therapeutic response in the treatment of leukemia, such as diminishing or eliminating leukemic cells, and/or their neoplastic precursors in the subject or preventing the further proliferation of leukemic cells, and/or their neoplastic precursors, in the subject. The amount required may vary from subject to subject, depending on the species, age, and general condition of the subject, the severity of the condition being treated, the particular agent or agents employed, the mode of administration, and the like. Thus, it may not be possible to specify an exact “effective amount”. However, an appropriate “effective amount” in any individual case may be determined by one of ordinary skill in the art using only routine experimentation. In some embodiments, an effective amount for a human subject lies in the range of about 0.1 ng/kg body weight/dose to 1 g/kg body weight/dose. In some embodiments, the range is about 1 μg to 1 g, about 1 mg to 1 g, 1 mg to 500 mg, 1 mg to 250 mg, 1 mg to 50 mg, or 1 μg to 1 mg/kg body weight/dose. Dosage regimes are adjusted to suit the exigencies of the situation and may be adjusted to produce the optimum therapeutic dose. For example, several doses may be provided daily, weekly, monthly or other appropriate time intervals.

The agent taught herein may be administered in a number of ways depending upon whether local or systemic treatment as desired. Examples of suitable routes of administration include intravenous, intra-arterial, subcutaneous, intraperitoneal or intramuscular injection or infusion, oral administration or via a spinal tap. In an embodiment disclosed herein, the agent is administered intravenously.

In an embodiment disclosed herein, the agent is an immuno-interactive molecule. An immuno-interactive molecule, in the context of the present disclosure, is a molecule capable of binding to a leukemic cell that expresses Fat1. The molecule may bind to Fat1 expressed on the leukemic cell, or it may bind to a companion marker (i.e., another marker that is co-expressed by Fat1-expressing leukemic cells). In an embodiment disclosed herein, the immuno-interactive molecule is an agent that specifically binds to Fat1 on the surface of a leukemic cell.

In an embodiment, the immuno-interactive molecule is an antibody or Fat1-binding fragment thereof. Antibodies suitable for use in accordance with the methods disclosed herein would be known to those skilled in the art. Examples include, but are not limited to, polyclonal, monoclonal, mono-specific, poly-specific (including bi-specific), humanized, single-chain, chimeric, synthetic, recombinant, hybrid, mutated, and CDR-grafted antibodies. Various techniques for producing antibodies and preparing recombinant antibody molecules are known in the art. Antibodies may be derived from any species, including, but not limited to, rat, mouse, goat, guinea pig, donkey, rabbit, horse, lama, camel, or any avian species (e.g., chicken, duck). The antibody may be of any suitable isotype, such as IgG, IgM, IgA, IgD, IgE or any subclass thereof. The skilled addressee will appreciate that antibodies produced recombinantly, or by other means, for use in accordance with the methods embodied herein include fragments that are still capable of binding to or otherwise recognizing Fat1 on a leukemic cell, a neoplastic precursor thereof. Examples include Fab, an F(ab)₂, Fv, scFv fragments.

In an embodiment, the antibody is a monoclonal antibody or a Fat1-binding fragment thereof. The monoclonal antibody can be a humanised or deimmunised form of a non-human antibody. In another embodiment, the monoclonal antibody is a human antibody.

In an embodiment disclosed herein, the immuno-interactive molecule is a multi-specific antibody that is capable of specifically binding to at least two antigens on a leukemic cell or its neoplastic precursor, wherein one of the at least two antigens is Fat1 or a homolog thereof. The multi-specific antibody may a bi-specific antibody that binds to Fat1 or a homolog thereof.

In an embodiment disclosed herein, the immuno-interactive molecule is labeled with a cytotoxic moiety. Suitable cytotoxic moieties are known to those skilled in the art. Examples include a toxin, an apoptotic agent or a radioactive isotope. In another embodiment, the immuno-reactive molecule is an antibody that is cytotoxic to the cell by complement-directed means.

Where necessary, the method disclosed herein may further comprise administrating to the subject in need thereof another anti-cancer agent. The other anti-cancer agent may be administered to the subject in need thereof sequentially (before or after administration of the agent disclosed herein) or concurrently.

In another aspect, there is provided use of an agent that is selectively cytotoxic to leukemic cells or neoplastic precursors thereof that express Fat1, or a homolog of Fat1 that is substantially not expressed on normal blood cells, in the manufacture of a medicament for the treatment of a subject with leukemia.

In an embodiment disclosed herein, the medicament is formulated for administration with another anti-cancer agent.

In another aspect, there is provided a composition comprising an agent that is selectively cytotoxic to leukemic cells or neoplastic precursors thereof that express Fat1, or a homolog of Fat1 that is substantially not expressed on normal blood cells, and one or more pharmaceutically acceptable carriers, diluents or excipients.

By pharmaceutically acceptable carrier, diluent or excipient is meant a pharmaceutical vehicle comprised of a material that is not biologically or otherwise undesirable, i.e. the material may be administered to a subject along with the selected conjugate without causing any or a substantial adverse reaction. Carriers may include excipients and other additives such as diluents, detergents, colouring agents, wetting or emulsifying agents, pH buffering agents, preservatives, and the like. Carriers may also include all conventional solvents, dispersion media, fillers, solid carriers, coatings, antifungal and antibacterial agents, dermal penetration agents, surfactants, isotonic and absorption agents and the like. It will be understood that the compositions of the invention may also include other supplementary physiologically active agents.

Compositions of the present invention suitable for oral administration may be presented as a solution or a suspension in an aqueous or non-aqueous liquid; or as an oil-in-water liquid emulsion or a water-in-oil liquid emulsion.

Also enabled herein are pharmaceutical compositions and formulations which include one or more additional anti-cancer agents. The pharmaceutical compositions taught herein may be administered in a number of ways depending upon whether local or systemic treatment as desired. Administration includes intravenous, intra-arterial, subcutaneous, intraperitoneal or intramuscular injection or infusion, oral administration or via a spinal tap. Conventional pharmaceutical carriers, aqueous, powder or oily bases, thickeners and the like may be necessary or desirable.

The pharmaceutical formulations described herein may conveniently be presented in unit dosage form and may be prepared according to conventional techniques well known in the pharmaceutical industry. Such techniques include the step of bringing into association the active agent(s) with the pharmaceutical carrier(s) or excipient(s).

The compositions described herein may be formulated into any of many possible dosage forms such as, but not limited to, injectable formulations, and tablets, capsules, gel capsules and liquids.

Pharmaceutical compositions herein include, but are not limited to, solutions, emulsions, foams and liposome-containing formulations. The pharmaceutical compositions and formulations herein described may comprise one or more penetration enhancers, carriers, excipients or other active or inactive ingredients.

Emulsions are typically heterogeneous systems of one liquid dispersed in another in the form of droplets usually exceeding 0.1 μm in diameter. Emulsions may contain additional components in addition to the dispersed phases, and the active drug which may be present as a solution in either the aqueous phase, oily phase or itself as a separate phase. Microemulsions are included as an embodiment taught herein.

In an embodiment disclosed herein, a penetration enhancer may be employed to enhance the delivery of agent to the subject in need thereof. In addition to aiding the diffusion of non-lipophilic agents across cell membranes, penetration enhancers may also enhance the permeability of lipophilic drugs. Penetration enhancers may be classified as belonging to one of five broad categories, i.e., surfactants, fatty acids, bile salts, chelating agents, and non-chelating non-surfactants.

One of skill in the art will recognize that formulations are routinely designed according to their intended use (i.e. route of administration).

The formulation of the composition and its subsequent administration (dosing) are within the skill of those in the art. Dosing is dependent on severity of disease and responsiveness of the subject to treatment, with the course of treatment lasting from several days to several months, or until a cure is effected or a diminution of the disease state is achieved (e.g., relapse). Optimal dosing schedules can be calculated from measurements of drug accumulation in the body of the patient. Persons of ordinary skill can easily determine optimum dosages, dosing methodologies and repetition rates. In general, dosage is from 0.01 μg to 100 g per kg of body weight, and may be given once or more daily, weekly, monthly or yearly, or even once every 2 to 20 years. Persons of ordinary skill in the art can easily estimate repetition rates for dosing based on measured residence times and concentrations of the drug in bodily fluids or tissues. Following successful treatment, it may be desirable to have the patient undergo maintenance therapy to prevent relapse.

Aspects disclosed herein are based on the surprising findings that leukemic cells can be distinguished from non-leukemic cells by virtue of the differential expression of Fat1 cadherin on leukemic cells. Accordingly, in another aspect, there is provided a method for the diagnosis or prognosis of a leukemia in a subject, the method comprising executing the step of analyzing a blood sample from the subject for the presence of cells that express Fat1 or a homolog thereof that is substantially not expressed by normal blood cells, wherein the execution step comprises contacting the blood sample with a primary binding agent that is capable of specifically binding to Fat1, or a homolog thereof, on blood cells, wherein the binding of the primary binding agent to a cell is indicative of presence of a cell that expresses Fat1 or its homolog and provides an indication of the presence of a leukemic cell or a precursor thereof.

As used herein, reference to a binding agent that is capable of specifically binding to Fat1, or a homolog thereof includes reference to a binding agent that binds to Fat1, or a homolog thereof.

As used herein, the term “primary binding agent” means any substance that is capable of recognizing (i.e., binding to) Fat1 (or a homolog thereof) on a leukemic cell and that is then capable of subsequent detection. Suitable primary binding agents would be known to those skilled in the art and the choice will depend on the nature of the sample and the execution step. In an embodiment disclosed herein, the primary binding agent is an antibody, or a Fat1-binding fragment thereof, also referred to herein as a primary antibody. The primary binding agent may further comprise a functional element, including, but not limited to, a polymer and/or linker segment, a detectable label, and/or an element that may be recognized by an adaptor unit or detectable label.

The skilled person would understand that, where necessary, the method may comprise using a secondary binding agent to increase the sensitivity of the method. As used herein, the term “secondary binding agent” means any substance that is capable of binding to or otherwise recognizing the primary binding agent. Suitable secondary binding agents would be known to those skilled in the art. Examples include antibodies, or antigen binding fragments thereof, also referred to herein as secondary antibodies. Antibodies suitable for use as secondary binding agents would be known to those skilled in the art and include polyclonal, monoclonal, humanized, single-chain, chimeric, synthetic, recombinant, hybrid, mutated and CDR-grafted antibodies. Antibodies may be derived from any species, as hereinbefore described, and may be of any suitable isotype, such as IgG, IgM, IgA, IgD, IgE or any subclass thereof. The skilled addressee will appreciate that antibodies produced recombinantly, or by other means, for use in accordance with the present invention include antigen-binding fragments thereof that can still bind to or otherwise recognize the primary binding agent. Examples include Fab, an F(ab)₂, Fv, scFv fragments.

The terms “recognize”, “recognizing” and the like, as used herein, mean an event in which one substance, such as a binding agent, directly or indirectly interacts with a target molecule in such a way that the interaction with the target may be detected. In some examples, a binding agent may react with a target, or directly bind to a target, or indirectly react with or bind to a target by directly binding to another substance that in turn directly binds to or reacts with a target. The terms “specific for”, “specifically” and the like, as used herein in the context of describing binding between two or more entities, mean that the binding is through a specific interaction between complementary binding partners, rather than through non-specific aggregation.

Suitable detectable labels are known to those skilled in the art. Examples include any molecule that may be detected directly or indirectly so as to reveal the presence of a target (e.g., Fat1) on a cell. Examples of detectable labels which may be used in accordance with the present invention include fluorophores, radioactive isotopes, chromophores, electrochemiluminescent labels, bioluminescent labels, polymers, polymer particles, beads or other solid surfaces, gold or other metal particles or heavy atoms, spin labels, haptens, myc, nitrotyrosine, biotin and avidin. Others include phosphor particles, doped particles, nanocrystals or quantum dots.

In an embodiment disclosed herein, a direct detectable label is used. Direct detectable labels may be detected per se without the need for additional molecules. In another embodiment, an indirect detectable label is used, which requires the employment of one or more additional molecules so as to a form detectable molecular complex (e.g., a biotin-avidin complex).

In another aspect, there is provided a method for the diagnosis or prognosis of a leukemia in a subject, the method comprising executing the step of analyzing a blood sample from the subject for the presence of cells that express Fat1 or a homolog thereof that is substantially not expressed by normal blood cells, wherein the execution step comprises contacting nucleic acid from the blood sample with an oligonucleotide probe that is capable of hybridizing to a nucleic acid sequence encoding Fat1, or a homolog thereof, wherein the binding of the probe to the nucleic acid from the blood sample is indicative of presence of a cell that expresses Fat1 or its homolog and provides an indication of the presence of a leukemic cell or a precursor thereof.

As used herein, reference to an oligonucleotide probe that is capable of hybridizing to a nucleic acid sequence encoding Fat1, or a homolog thereof includes reference to an oligonucleotide probe that hybridizes to a nucleic acid sequence encoding Fat1, or a homolog thereof.

In an embodiment of the present disclosure, the nucleic acid from the blood sample is mRNA encoding Fat1 or a homolog thereof. The nucleic acid may be isolated from a blood sample using methods known to those skilled in the art. Isolation of a nucleic acid is to be understood to mean a nucleic acid that has generally been separated from other components with which it is naturally associated or linked in its native state. In an embodiment, the isolated nucleic acid is at least 50% free, preferably at least 75% free, and more preferably at least 90% free from other components with which it is naturally associated. The degree of isolation expressed may relate to purity from interfering substances.

In an embodiment disclosed herein, the binding of the probe to a nucleic acid sequence encoding Fat1 or a homolog thereof is detected using a detectable label. The detectable label, such as those described herein, may be attached to the probe itself.

In another embodiment, the presence of a nucleic acid sequence encoding Fat1 or a homolog thereof can be detected by amplifying the specific sequence to which the probe has hybridized. Suitable amplification methods are known to those skilled in the art. Examples include isolating mRNA from the blood sample, reverse transcribing the mRNA into cDNA and using a sequence-specific probe to amplify a nucleic acid sequence encoding Fat1 or a homolog thereof using reverse transcription-polymerase chain reaction (RT-PCR).

The presence of a nucleic acid sequence encoding Fat1 or a homolog thereof may also be detected in a blood sample using array-based technology. Array-based technologies are known to those skilled in the art and include microarrays, DNA microarrays, DNA chips, hybridisation arrays and the like. An array-based technology will typically comprise a solid support typically having nucleotide probes (e.g., oligonucleotide probes) arrayed on its surface. The solid support utilised in the preparation of a chip or microarray may be a nitrocellulose or nylon membrane, or a glass, plastic or silicon slide, or a bead. An array may comprise an ordered arrangement of hybridisable array elements, wherein at least one array element is an oligonucleotide probe that is capable of specifically hybridizing to a nucleic acid sequence encoding Fat1 or a homolog thereof. The array elements can be arranged so that there are multiple copies of a single element as an internal control, enough copies of positive and negative controls to determine background hybridisation. One or more different array elements may be immobilised to a substrate surface. In an embodiment disclosed herein, at least 10 array elements are immobilised to a substrate surface. In an embodiment, at least 100 array elements are immobilised to a substrate surface. In an embodiment, at least 5,000 array elements are immobilised to a substrate surface. Where an array surface is small, for example 1 cm², the array may be referred to as a “microarray”. Furthermore, the hybridisation signal from respective array elements is individually distinguishable.

In another embodiment, the execution step may be performed without prior isolation of the nucleic acid. For example, mRNA encoding Fat1 or a homolog thereof can be contacted with the probe in situ (e.g., by in situ hybridization).

In another aspect, there is provided a therapeutic protocol for treating leukemia in a subject, said protocol comprising the steps of:

a. executing the step of analyzing a sample of blood from the subject for the presence of cells that express Fat1 or a homolog thereof that is substantially not expressed on normal blood cells, wherein the execution step comprises contacting a primary binding agent that is capable of specifically binding to Fat1 on a blood cell, wherein the presence of a cell that expresses Fat1 is indicative of the presence of a leukemic cell or a precursor form thereof;
b. administering to a subject who contains Fat1-expressing cells an agent that is selectively cytotoxic to leukemic cells, or neoplastic precursors thereof that express Fat1 or a homolog of Fat1 that is substantially not expressed on normal blood cells; and
c. monitoring for a reduction in the presence of Fat1-expressing cells over time;
wherein a reduction in Fat1-expressing cells over a period of time is indicative of a successful treatment.

In another aspect, there is provided a therapeutic protocol for treating leukemia in a subject, said protocol comprising the steps of:

d. executing the step of analyzing a sample of blood from the subject for the presence of cells that express Fat1 or a homolog thereof that is substantially not expressed on normal blood cells, wherein the execution step comprises contacting nucleic acid from the blood sample with a probe that is capable of hybridizing under stringent conditions to a nucleic acid sequence encoding Fat1, or a homolog thereof, wherein the binding of the probe to the nucleic acid from the blood sample is indicative of presence of a cell that expresses Fat1 or its homolog and provides an indication of the presence of a leukemic cell or a precursor thereof;
e. administering to a subject who contains Fat1-expressing cells an agent that is selectively cytotoxic to leukemic cells, or neoplastic precursors thereof that express Fat1 or a homolog of Fat1 that is substantially not expressed on normal blood cells; and
f. monitoring for a reduction in the presence of Fat1-expressing cells over time;
wherein a reduction in Fat1-expressing cells over a period of time is indicative of a successful treatment.

In another aspect, there is provided a method of vaccinating a subject against leukemia, the method comprising administering to the subject an amount of a compound comprising a Fat1 polypeptide, or a immunogenic fragment thereof, effective to stimulate antibodies against Fat1 expressed by cells in the subject.

In an embodiment disclosed herein, the compound is administered with an immunogenic carrier. Suitable carriers would be known to those skilled in the art. Examples include emulsifiers, muramyl dipeptides, avridine, aqueous adjuvants such as aluminum hydroxide, chitosan-based adjuvants, and any of the various saponins, oils, and other substances known in the art, such as amphigen, LPS, bacterial cell wall extracts, bacterial DNA, CpG sequences, synthetic oligonucleotides and combinations thereof.

Those skilled in the art will appreciate that aspects described herein are susceptible to variations and modifications other than those specifically described. It is to be understood that these aspects include all such variations and modifications. The disclosure also includes all of the steps, features, compositions and compounds referred to or indicated in this specification, individually or collectively, and any and all combinations of any two or more of the steps or features.

### EXAMPLES

Aspects taught herein are further described by the following non-limiting Examples. In these Examples, materials and methods as outline below are employed.

### Materials and Methods

### A. Cell Culture

Human leukemic T-cell ALL (Jurkat, HPB-ALL and MOLT-4), preB-ALL (NALM-6 and LK63), B-ALL (BALM-1) and AML (THP-1 and R2CA) were all maintained in RPMI 1640 media supplemented with 10% fetal bovine serum (Trace Biosciences, Castle Hill, NSW, Australia), 20 mM HEPES (4-(2-hydroxyethyl)-1-piperazineethanesulfonic acid), 2 mM Glutamax and 2% penicillin-streptomycin (all from Invitrogen, Carlsbad, Calif., USA) and were cultured in a humidified incubator at 37° C. with 5% CO₂.

### B. Western Blot Analysis

Suspension cells were harvested bye centrifugation and washed twice with ice-cold phosphate-buffered saline, lysed in NDE lysis buffer (10 mM Tris-HCl, 1% NP40, 0.4% sodium deoxycholate and 66 mM EDTA). Electrophoresis on NuPAGE 3-8% Iris-Acetate gels (Invitrogen) and Western blotting were carried out as previously described (Sadeqzadeh et al., 2011).

### C. In Silico and Statistical Analysis of Microarray Data Sets

The CEL files from publicly available microarray gene expression data sets from NCBI's gene expression omnibus (GEO) database (http://www.ncbi.nlm.nih.gov/geo/) were analyzed using MAS5.0 algorithm (Expression Console software VI A, Affymetrix, Santa Clara, Calif., USA). Raw data were analyzed using the MAS5.0 algorithm to generate an absolute call and associated P-value. The presence of the Fat1 transcript was defined as a P-value<0.06, and absent with a P-value>0.06, reflecting the P-values. Correlation of relative Fat1 expression within applicable data sets with patient outcome was carried out using R software with additional Bioconductor packages (http://www.r-project.org and http://www.bioconductor.org). The primary end points for the survival analyses was either disease-specific survival or relapse-free survival, which was measured from the date of diagnosis to disease-specific death or first relapse, or otherwise censored at the time of the last follow-up visit or at non-disease-related death, Time to first relapse or disease-specific death was plotted as Kaplan-Meier survival curves. Cox proportional hazards regression was used for univariate analysis of the prognostic impact of Fat1 expression. For statistical analysis, SPSS (Version 15.0.1; SPSS Inc., Chicago, Ill., USA) software was used.

D. Isolation of HSC from Hone Marrow and Peripheral Blood

Peripheral blood (PB) hematopoietic stem cells (HSCs) were isolated using fluorescence-activated cell sorting from samples obtained from therapeutic; donors with their informed consent (for example, polycythemic patients) using CD34 and CD133 cell surface markers. Buffy coats were first obtained from whole blood bags after centrifugation at 400 g for 10 min and then RBC removed using the Dextran 500 method sedimentation. WBCs were then washed and overlaid onto Ficoll-Paque (#15-5442-02, GE Healthcare, NSW, Australia) to collect MNCs that were then further enriched by removing Lin+ cells (progenitor enrichment kit (EasySep® Human Progenitor Cell Enrichment Kit #19056, Stem Cell Technologies, NSW Australia). Negatively isolated cells were then triple antigen stained with CD34-PE (130-081-002), CD133-APC (130-090-854) and CD45-VioBlue (130-092-880) (Miltenyi Biotec, NSW Australia) along with 7-AAD (A1310-Invitrogen) to enable dead cell discrimination, CD34+ HSCs were then sorted using the ISHAGE approach on a FACS Aria H instrument (Becton Dickinson). HSCs from commercial normal hone marrow (BM) mononuclear cells (no. 2M-25D; Lonza, VIC, Australia) were prepared using positive immunomagnetic enrichment. Briefly, anti-CD34 (no. CBL496; Chemicon International) or CD133 (no. 130-090-422, Miltenyi Biotec, NSW Australia) monoclonal antibodies (mAbs) were used to prepare bispecific tetrameric antibody complexes and antigen-positive cells from BM isolated according to manufacturer's instructions (Ease/Sep, Stem Cell Technologies, NSW, Australia). To validate the successful isolation of functional HSCs in the isolated populations, cells were inoculated into methylcellulose media (no. 130-091-280; Miltenyi Biotec) to confirm their in vitro clonogenic capacity.

### E. Real-Time Quantitative PCR Analysis

Total RNA was isolated and reverse transcribed to complementary DNA using the Illustra RNAspin isolation Kit (GE Healthcare) and Transcriptor High Fidelity cDNA synthesis kit (Roche Diagnostics), respectively. The Applied Biosystems 7500 Real-Time PCR System was used to compare the expression level of Fat1 with that of GusB and ABL housekeeping controls. For patient BM samples, quantitative PCR (qPCR) was carried out as described previously (Ponassi et al., 1999) using the primer sequences in Table 6, below.

F. BM Samples from Patients with Acute Lymphoblastic and AML Leukemia

BM samples from ALL and AML patients were sourced from the Tumour Bank at The Children's Hospital at Westmead, with BM collected at the time of diagnosis. Informed consent was obtained from the parents of all patients according to the regulations of The Children's Hospital at Westmead Ethics Committee.

### Example 1

### Fat1 Expression is Found in Leukemia Cell Lines but not in PB Cells or Enriched Hematopoietic Progenitor Cells

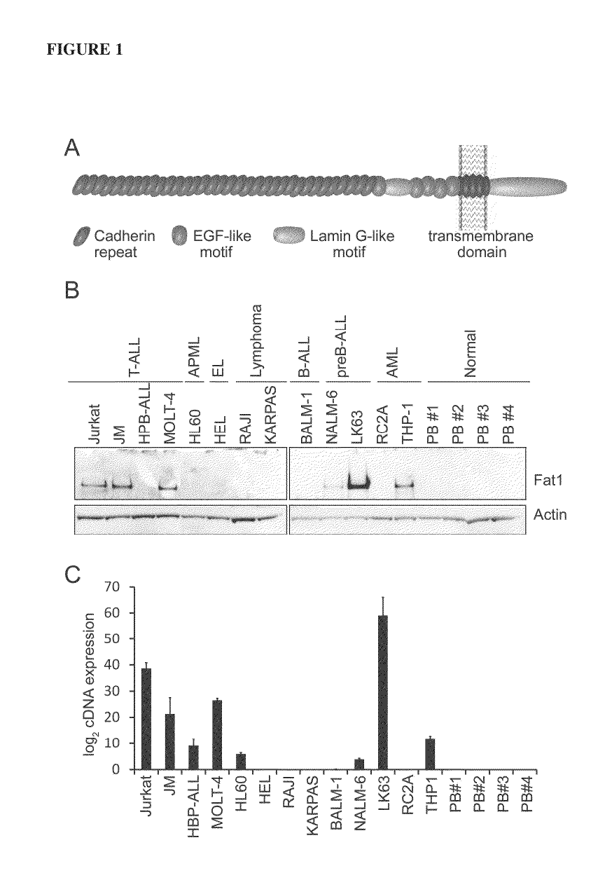

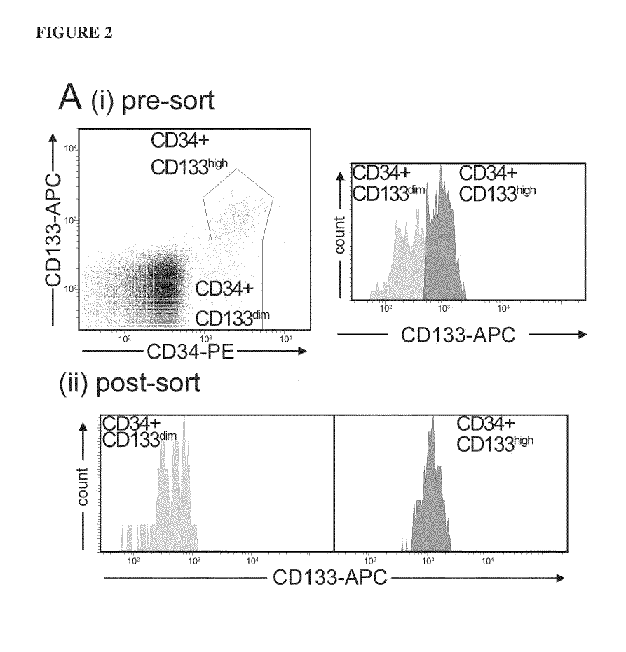

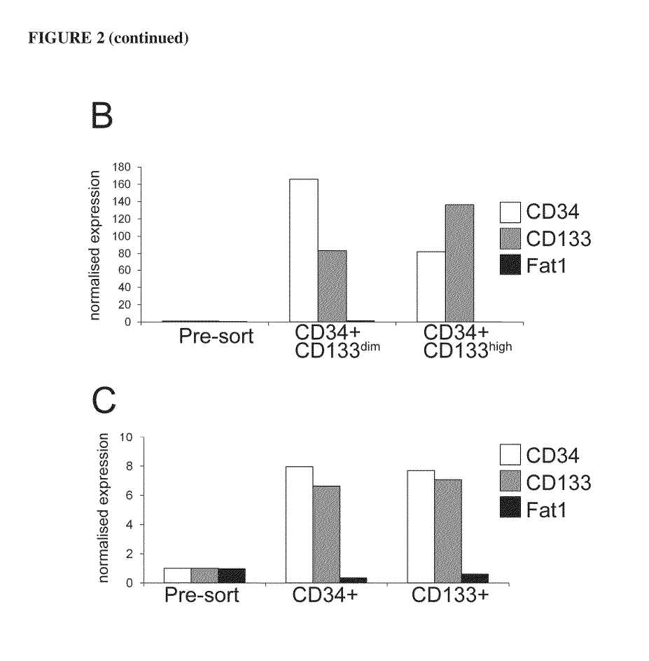

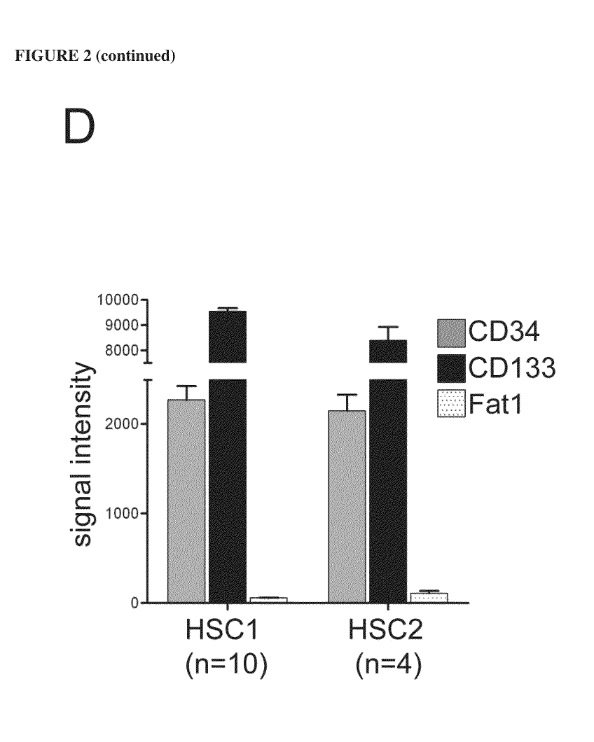

The Fat1 cadherin is a type I transmembrane protein with 34 cadherin repeats, 5 epidermal growth factor-like repeats and 1 laminin G motif on the extracellular side of the cell, followed by a transmembrane region and cytoplasmic domain (FIG. 1a). Initial analysis of Fat1 expression focused on a leukemic cell line panel and normal PB cells from four healthy donors using western blotting and qPCR. In the Western blot, the preB- and B-ALL cell lines LK63 and Nalm-6, AML cell line THP-1 and T-ALL cell lines Jurkat, JM and MOLT-4 all showed an immunoreactive band for Fat1 resolving at ˜550 kDa. The remaining cell lines and PB mononuclear cells from four healthy donors showed no immunoreactivity in this region indicative of either no Fat1 expression or a level of expression below detection limit (FIG. 1b). The levels of the protein largely reconciled with the level of Fat1 mRNA transcript as measured by qPCR, with the exception of HPB-ALL and HL-60, both of which had significant Fat1 mRNA signal but no discernable full-length Fat1 protein expression as detected by western blot (FIG. 1c). The expression of vertebrate Fat1 cadherin in mouse and rat has previously been shown to be high during development (Ponassi et al., 1999; Cox et al., 2000). It therefore remained to be determined whether Fat1 may be enriched in hematopoietic progenitors. To this end, CD34⁺/CD133^{dim} and CD34⁺/CD133^{bright} cell were sorted and validated from the circulating PB of non-leukemic patients using flow cytometry (FIG. 2a), and then analyzed by qPCR to determine the levels of Fat1 mRNA within these two defined populations. The qPCR results show that there was no significant expression of Fat1 mRNA in either of the enriched CD34⁺/CD133^{dim} and CD34⁺/CD133^{bright} cell populations (FIG. 2b). Utilizing an independent method, CD34+ and CD133+ cells were enriched from the BM of non-leukemic patients using magnetic beads and analyzed by qPCR for Fat1 mRNA expression. This analysis also demonstrated that there was no enrichment of Fat1 associated with hematopoietic progenitor populations, as there was less Fat1 transcript in the enriched population compared with presorted control population of cells (FIG. 2c).

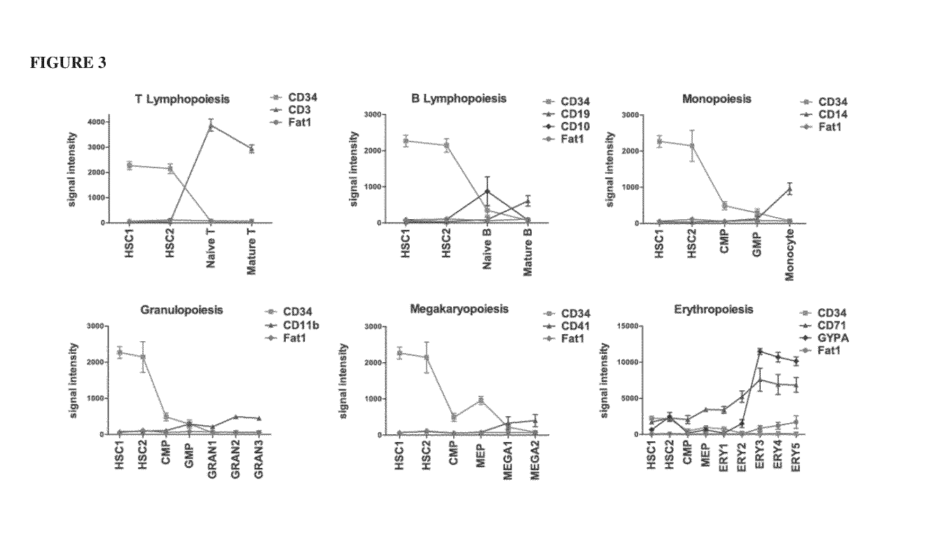

To extend these studies and determine whether Fat1 was expressed at any significant level during normal hematopoiesis, the level of Fat1 transcript was analyzed by gene expression profiling performed in multiple stages of hematopoietic differentiation (GSE24759). In this study, 38 subpopulations representing different lineages and maturation states were obtained using multiparameter fluorescence-activated cell sorting and profiled using HG_U133AAofAv2 microarrays (Affymetrix). Fat1 transcript levels were examined in silico using the deposited log₂-transformed normalized data. Consistent with the fluorescence-activated cell sorting data disclosed herein, there was no significant Fat1 transcript (P<0.05) in their HSC1 (Lin⁻, CD133⁻ and CD34^{dim}) or HSC2 CD38⁻ and CD34⁺) population (FIG. 2d). The same data was then used to determine the extent and level of Fat1 expression across six separate lineage signatures (FIG. 3). From this analysis, significant but relatively low Fat1 transcript expression occurred only within the erythroid lineage and, in particular, in the defined early and late erythroid signatures. Here, Fat1 in the early erythroid signature ranked 131/1228 (P-value=L55249×10⁻²⁶), and within the late erythroid signature ranked 149/1270 (P-value=5.34305×10⁻⁴³). To determine whether red blood cells in the peripheral blood (PB) express Fat1 protein, immunoprecipitation was carried out, but Fat1 was unable to be detected in circulating red blood cells.

### Example 2

### In Silico Analysis of Clinical Microarray Data Reveals that Fat1 mRNA Transcript is Present in AML, BALL and T-ALL but not in Normal Blood Cells and their Progenitors

Publicly available microarray data deposited within the GEO or similar databases using Affymetrix-based platforms were mined to determine the number of cases where Fat1 transcript is present within cohorts of clinically relevant leukemia samples, including AML, B-ALL, T-ALL and normal PB and BM cells. The MAS5.0 algorithm used (see Materials and Methods) generates three distinct calls (present, marginal or absent) for each probe present on the array (Affymetrix ID), with empirical threshold probability values of P<0.06, including both marginal and present calls, and a P-value>0.06 classified as absent. For this study, the extent of Fat1 expression was determined (significance set for both present and marginal) in B-ALL, T-ALL and AML, for the purpose of examining the applicability of Fat1 as a unique leukemia target compared with normal blood cells and their progenitors. The significant detection of Fat1 transcript in eight separate AML array studies is presented in Table 1, with the sum of all cases resulting in Fat1 transcript present in 11% of cases. In eight separate B-ALL array studies, the sum of all cases shows that Fat1 transcript is present in 29% of cases (Table 2); and for eight T-ALL array studies the sum of all cases shows that Fat1 transcript is present in 63.5% (Table 3). A total of six separate analysis incorporating different subsets of nominal blood cells was then analyzed (Table 4). In a study looking at early hematopoietic cell progenitors isolated from either umbilical cord blood or the BM (Eckfeldt et al., 2005), only one out of four cases of BM cells enriched for CD34⁺CD38^{-CD33-}Rho^{high} had a significant signal for Fat1 transcript. In a second study analyzing total PB (Valk et al., 2004), only one case out of five had a significant signal for Fat1 transcript. These data showing the lack of significant Fat transcript signals in the majority of healthy normal blood cells and their progenitors reconcile with the findings herein of low expression levels of Fat1 transcript from CD34⁺- and CD133⁺-isolated HSCs (FIGS. 2 and 3).

### Example 3

### Fat1 Transcript can be Detected by VCR in Clinically Relevant Leukemia BM Aspirate Samples

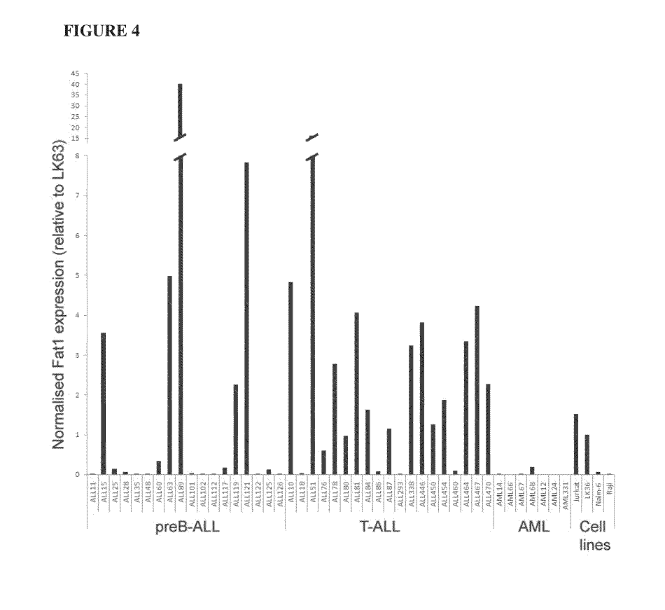

To extend the in silico analysis and directly verify the presence of Fat1 transcript in clinically relevant pediatric leukemia samples, a cohort of 18 preB-ALL, 19 T-ALL and 7 AML pediatric BM samples (patient characteristics and phenotypes listed in Table 7 and Table 8 were assessed for Fat1 using qPCR. The clinical samples were also directly compared in the same assays with representative cell lines expressing high levels of Fad (Jurkat I-ALL and LK63 preB-ALL), low levels of Fat1 (Nalm-6 preB-ALL) and Fat1-negative cells (Raji lymphoma). The qPCR analyses of clinical samples (FIG. 4) were normalized to LK63 (high Fat1; set as 1) and with an arbitrary cutoff equivalent to Nalm-6 (low Fat1; Fat1 qPCR signal 2^{-Ct}>0.06; Table 7 and Table 8.). This criterion was then used to assess the number of Fat1-positive cases' results with 10/18 Fat1 positive for B-ALL, 18/19 Fat1 positive for T-ALL and Fat1 1/7 positive for AML. Although this clinical cohort used was small, the overall trend for Fat1-positive clones across the different leukemia phenotypes (T-ALL>B-ALL>AML).

### Example 4

### Fat1 Expression is Prognostic for Disease Relapse and Overall Survival in Pediatric preB-ALL in Paired Diagnosis-Relapse Samples

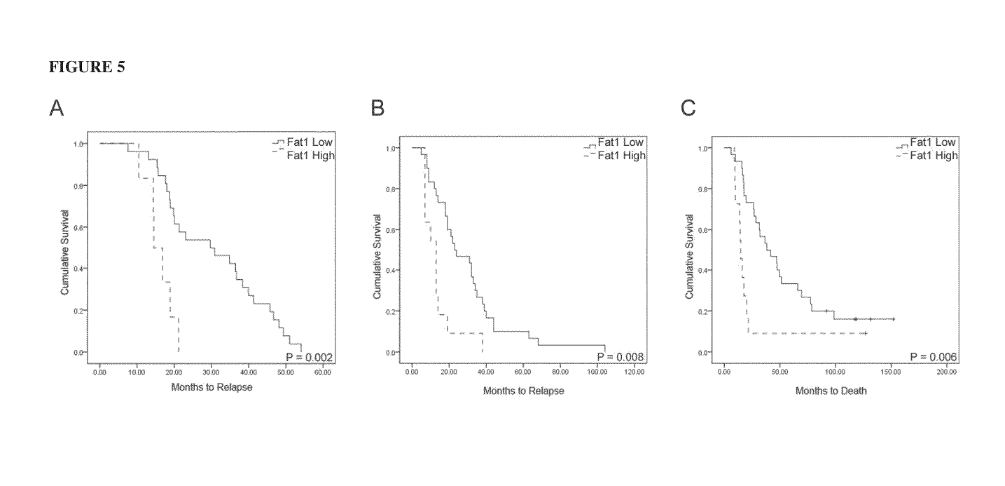

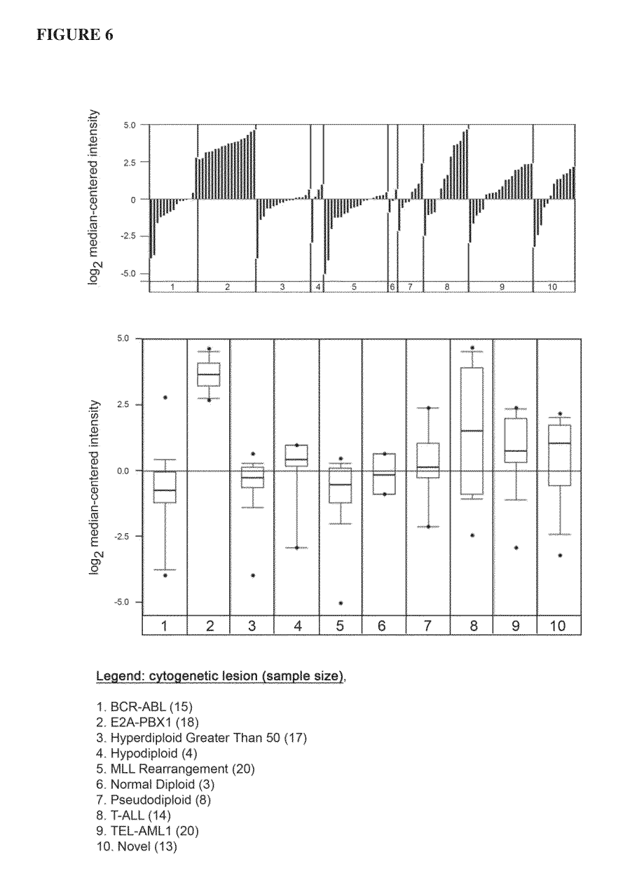

For relevant microarray data sets in which patient outcome data were available, the level of Fat1 expression was assessed as a predictor of patient outcome for ALL subsets. For preB-ALL, two recently published array sets (Bhojwani et al., 2006; Staal et al., 2010) with matched pediatric diagnosis-relapse patients were analyzed. The use of matched diagnosis-relapse samples affords several advantages over a conventional cohort; it not only lends itself to the identification of genetic pathways and molecular mechanisms involved in relapse but also provides insights into the origins of the relapsed clone. Both are important aspects for consideration in identifying putative targets for future therapies. For the matched preB-ALL patients, the primary end points for the survival analyses was either disease-specific survival or relapse-free survival, which was measured from the date of diagnosis to disease-specific death or first relapse, or otherwise censored at the time of the last follow-up visit or at non-disease-related death. For the 32 preB-ALL patients, the Fat1 signal intensity assessed by the MAS5.0 algorithm ranged from 58 to 1683 (mean=304) at the time of diagnosis, and ranged from 21 to 3630 (mean=363) at the time of relapse. High Fat1 expression at the time of diagnosis (upper quartile cutoff) had significantly increased risk of relapse compared with lower Fat1 levels of expression (remaining 75%; hazard ratio=5.09; 95% confidence interval 1.80-14.41; P=0.002; FIG. 5), with a median relapse-free survival of 15.3 months compared with 30.1 months, respectively.

Univariate analysis showed high Fat1 expression at diagnosis (upper quartile cutoff) compared with lower Fat1 levels of expression (remaining 75%), which had significantly shortened relapse-free (median=13 versus 23.5) and overall survival (median=14.5 versus 32) (FIG. 5, Table 5). Multivariate analysis against other criteria of risk assessment, including age, white blood cell count and sex showed that Fat1 is an independent prognostic marker for relapse-free survival and overall survival in preB-ALL (Table 5).

TABLE 1
In silico analysis of acute myeloid leukemia samples
for the presence of the Fat1 transcript.

| | AML | Cases Fat1 transcript | |
|---|---|---|---|
| GEO/source | Subtype/Sample | present (p < 0.06) | % |
| GSE14468 | | 52/525 | 10% |
| GSE1159 | | 40/285 | 14% |
| GSE14471 | | 8/111 | 7% |
| GSE15434 | Normal Karyotype | 30/251 | 12% |
| GSE12417 | Normal Karyotype | 23/163 | 14% |
| GSE12326 | CD34+ paired (BM | 0/10 | 0% |
| | and PB) | | |
| GSE9476 | BM and PB | 3/26 | 12% |
| GSE17061 | M0 | 4/35 | 11% |

| | | |
|---|---|---|
| SUM EXPRESSION | 160/1406 | 11.4% |

TABLE 2
In silico analysis of B-cell acute lymphoblastic leukemia
samples for the presence of the Fat1 transcript.

| | Cases Fat1 transcript | |
|---|---|---|
| GEO/source | present (p < 0.06) | % |
| GSE3912 | 32/105 | 30% |
| GSE18497 | 13/54 | 24% |
| GSE4698 | 15/51 | 29% |
| GSE13425 | 34/154 | 22% |
| http://www.stjuderesearch.org/data/ALL1 | 93/286 | 33% |
| GSE7440 | 27/99 | 27% |
| GSE635 | 35/145 | 24% |
| GSE11877 | 74/220 | 34% |
| SUM EXPRESSION | 323/1114 | 29% |

TABLE 3
In silico analysis of T-cell acute lymphoblastic leukemia
samples for the presence of the Fat1 transcript.

| | T-ALL | Cases Fat1 transcript | |
|---|---|---|---|
| GEO/source | Subtype/Sample | present (p < 0.06) | % |
| GSE3912 | | 5/10 | 50% |
| GSE18497 | | 23/28 | 82% |
| GSE4698 | PreT-ALL | 1/6 | 17% |
| | | 1/2 | 50% |
| GSE13425 | | 23/36 | 64% |
| GSE635 | | 18/28 | 64% |
| GSE8879 | Atypical | 35/55 | 64% |
| stjuderesearch | | 29/45 | 64% |
| ALL1 | | | |
| GSE11877 | | 20/34 | 59% |

| | | |
|---|---|---|
| SUM EXPRESSION | 155/244 | 63.5% |

TABLE 4
In silico analysis of normal peripheral blood and bone
marrow samples for the presence of the Fat1 transcript.

| | | Cases Fat1 | |
|---|---|---|---|
| | | transcript | |
| | | present | |
| GEO/source | Normal PB/BM Subtype/Sample | (p < 0.06) | % |
| GSE1493 | lin+CD34+ (PB or BM) | 0/2 | 0% |
| | lin−CD34+ (PB or BM) | 0/2 | 0% |
| | lin−CD34− (PB or BM) | 0/2 | 0% |
| | CD34+CD38−CD33−Rho(lo)c-kit+ | 0/4 | 0% |
| | (BM) | | |
| GSE2666 | CD34+CD38−CD33−Rho(hi) (BM) | 1/4 | 25% |
| | CD34+CD38−CD33−Rho(lo)c-kit+ | 0/5 | 0% |
| | (UC) | | |
| | CD34+CD38-CD33−Rho(hi) (UC) | 0/5 | 0% |
| GSE1159 | Total (PB or BM) | 1/5 | 20% |
| | CD34+ purified from three patients | 0/3 | 0% |
| GSE10438 | CD34+ CD38− Lin− (UC) | 0/3 | 0% |
| | CD34+, CD38−, CD36− (UC) | 0/3 | 0% |
| | CD34+, CD38+ (UC) | 0/3 | 0% |
| | Whole Blood | 0/3 | 0% |
| GSE 14924 | T-cell CD4+ | 0/10 | 0% |
| | T-cell CD8+ | 0/10 | 0% |
| GSE9476 | CD34+ (BM or PB) | 0/8 | 0% |
| | Unselected BM or PB | 0/10 | 0% |

TABLE 5
Univariate and multivariate Cox proportional hazards regression
analysis of age, gender, white blood cell count and Fat1 expression
(at diagnosis) for relapse-free and overall survival in GEO
dataset GSE18497. High Fat1 expression was significantly prognostic
with respect to both relapse-free and overall survival and was
independent of other clinical variables.

| | | Relapse-free | | | Overall | | |
|---|---|---|---|---|---|---|---|
| | | survival | | | survival | | |
| | | | p- | 95% | | p- | 95% |
| | Clinical variable | HR | value | CI | HR | value | CI |
| Uni- | Fat1 (Upper | 3.0 | 0.008 | 1.4-6.3 | 2.9 | 0.006 | 1.4-6.2 |
| variate | quartile vs. rest) | | | | | | |
| | Age (≧10 vs. <10) | 1.5 | 0.291 | 0.7-3.0 | 1.7 | 0.177 | 0.8-3.6 |
| | Gender (female | 0.8 | 0.471 | 0.4-1.6 | 0.8 | 0.631 | 0.4-1.8 |
| | vs. male) | | | | | | |
| | WBC (<50 × 10⁹/L | 2.1 | 0.028 | 1.1-4.1 | 1.4 | 0.292 | 0.7-2.8 |
| | vs. ≧50 × 10⁹/L) | | | | | | |
| Multi- | Fat1 (Upper | 2.8 | 0.007 | 1.3-5.9 | 2.9 | 0.007 | 1.3-6.2 |
| variate | quartile vs. rest) | | | | | | |
| | Age (≧10 vs. <10) | * | * | * | * | * | * |
| | Gender (female | * | * | * | * | * | * |
| | vs. male) | | | | | | |
| | WBC (<50 × 10⁹/L | * | * | * | * | * | * |
| | vs. ≧50 × 10⁹/L) | | | | | | |

* = not significant in final multivariate model.

TABLE 6
qPCR primers

| Primer | Sequence (5′→3′) | SEQ ID NO: |
|---|---|---|
| Fat1_Forward | GTG TGA TTC GGG TTT TAG GG | 3 |
| Fat1_Reverse | CTG TAC TCG TGG CTG CAG TT | 4 |
| CD34_Forward | GTC TAC TGC TGG TCT TGG C | 5 |
| CD34_Reverse | CTC TGG TGG CTT GCA ACA TC | 6 |
| CD133_Forward | CTG TTG ATG TCT TTC TGT GTA GCT AC | 7 |
| CD133_Reverse | CAT TCG ACG ATA GTA CTT AGC CAG | 8 |
| GusB_Forward | GCC AAT GAA ACC AGG TAT CCC | 9 |
| GusB_Reverse | GCT CAA GTA AAC AGG CTG TTT TCC | 10 |
| ABL_Forward | TGG AGA TAA CAC TCT AAG CAT AAC TAA | 11 |
| | AGG T | |
| ABL_Reverse | GAT GTA GTT GCT TGG GAC CCA | 12 |
| Actin_Forward | GGC ATC CTC ACC CTG AAG TA | 13 |
| Actin_Reverse | CCA TCT CTT GCT CGA AGT CC | 14 |

TABLE 7
Clinical characteristics of patients used in QPCR analysis of Fat1 expression in preB-ALL and T-ALL

| | | | | | Age at | Initial | Initial | Initial | | |
|---|---|---|---|---|---|---|---|---|---|---|
| ALL | Immuno- | Phenotype | CNS | Mediastinal | Diagnosis | White Blood | Blast | Platelets | Blast | Fat1 QPCR signal |
| Number | phenotype | (FAB) | Involvement | Involvement | (yr) | Cell Count | Count | Count | Percent | (2\hat{ }DCt) |
| ALL11 | Precusor B | FAB L1 | Y | N | 12 | 760 | 707 | 30 | 93.03 | 0.015197734 |
| ALL15 | Precusor B | FAB L2 | N | N | 14 | 16.9 | 10 | 98 | 56.21 | 3.555370725 |
| ALL25 | Precusor B | FAB L2 | N | N | 6 | 4.7 | 2 | 418 | 40.43 | 0.13805675 |
| ALL28 | Precusor B | FAB L2 | N | N | 45 months | 253 | 240 | 23 | 94.86 | 0.067607792 |
| ALL35 | Precusor B | FAB L1 | N | N | 38 months | 332 | 305 | 22 | 91.87 | 0.000376952 |
| ALL48 | Precusor B | FAB L2 | N | N | 10 | 4.2 | 1 | 70 | 19.05 | 0.01946015 |
| ALL60 | Precusor B | FAB L1 | N | N | 44 months | 15.4 | 6 | 65 | 38.96 | 0.338368381 |
| ALL63 | Precusor B | FAB L1 | N | N | 5 | 39.1 | 21 | 201 | 52.94 | 4.981798489 |
| ALL89 | Precusor B | FAB L1 | N | N | 15 | 6.2 | 1 | 99 | 19.35 | 40.22442798 |
| ALL101 | Precusor B | FAB L1 | N | N | 26 months | 208.8 | 186 | 9 | 89.08 | 0.032803646 |
| ALL102 | Precusor B | FAB L1 | N | N | 9 | 25.7 | 21 | 11 | 80.93 | 0.000526967 |
| ALL112 | Precusor B | FAB L1 | N | N | 20 months | 15 | 12 | 62 | 78.67 | 0.007616444 |
| ALL117 | Precusor B | FAB L1 | N | N | 10 | 8.6 | 1 | 257 | 15.12 | 0.1676278 |
| ALL119 | Precusor B | FAB L1 | N | N | 12 | 20.3 | 5 | 23 | 23.15 | 2.255321854 |
| ALL121 | Precusor B | FAB L1 | N | N | 12 | 24.9 | 22 | 34 | 89.96 | 7.835362381 |
| ALL122 | Precusor B | FAB L1 | N | N | 44 months | 13.5 | 9 | 130 | 69.63 | 0.004786986 |
| ALL125 | Precusor B | FAB L1 | N | N | 6 | 44 | 34 | 34 | 77.95 | 0.121301279 |
| ALL126 | Precusor B | FAB L1 | N | N | 36 months | 158.4 | 149 | 79 | 94 | 0.000208163 |
| ALL10 | T-Cell | FAB L1 | Y | N | 5 | 695 | 563 | 40 | 81.01 | 4.834388225 |
| ALL18 | T-Cell | FAB L2 | N | N | 5 | 45 | 15 | 86 | 33.33 | 0.039281668 |
| ALL51 | T-Cell | FAB L2 | N | Y | 11 | 96.8 | 74 | 181 | 76.03 | 16.22335168 |
| ALL76 | T-Cell | FAB L1 | N | Y | 6 | 119.3 | 56 | 83 | 46.94 | 0.595978972 |
| ALL78 | T-Cell | FAB L1 | N | N | 6 | 810 | 729 | 9 | 90 | 2.776626901 |
| ALL80 | T-Cell | | Y | N | 7 | 886 | 824 | 20 | 93 | 0.963707118 |
| ALL81 | T-Cell | FAB L1 | N | N | 11 | 2.6 | Occasional | 14 | 20-25 | 4.055837919 |
| ALL84 | T-Cell | FAB L1 | N | N | 13 | 333 | 290 | 34 | 87.09 | 1.624504793 |
| ALL86 | T-Cell | FAB L1 | N | N | 12 | 2.2 | 1 | 224 | 31.82 | 0.075712046 |
| ALL87 | T-Cell | FAB L1 | N | N | 15 | 142.8 | 113 | 28 | 78.99 | 1.159363791 |
| ALL293 | T-Cell | FAB L1 | N | Y | 31 months | 13.8 | 1 | 401 | 4.35 | 0.001953125 |
| ALL338 | T-Cell | FAB L2 | N | Y | 43 months | 38.9 | 13 | 206 | 32.9 | 3.234030609 |
| ALL446 | T-Cell | | N | N | 40 months | 177.4 | 149 | 90 | 83.9 | 3.810551992 |
| ALL450 | T-Cell | | N | N | 12 | 1.7 | 1 | 28 | 52.94 | 1.25411241 |
| ALL454 | T-Cell | | Y | N | 8 | 112.3 | 94 | 112 | 83.97 | 1.866065983 |
| ALL460 | T-Cell | | N | N | 14 | 196.3 | 158.3 | 131 | 80.64 | 0.097846677 |
| ALL464 | T-Cell | | Y | Y | 7 | 121.5 | 66 | 200 | 53.99 | 3.348078452 |
| ALL467 | T-Cell | FAB L1 | N | N | 4 | 19.8 | 10 | 29 | 47.98 | 4.237852377 |
| ALL470 | T-Cell | | N | N | 4 | 61.9 | 42 | 99 | 68.01 | 2.265767771 |
| AML331 | Biphenotypic | | | | | 12.2 | 12 | 18 | 90 | N/A |
| | Leukaemia - | | | | | | | | | |
| | relapsed AML | | | | | | | | | |

TABLE 8
Clinical characteristics of patients used in QPCR analysis
of Fat1 expression in AML (WBC = White Blood Cell)

| AML | Immuno- | | Age at | Initial | | | Fat1 QPCR signal |
|---|---|---|---|---|---|---|---|
| Number | phenotype | Morphology | Diagnosis (yr) | WBC Count | Cytogenetics | Status | (2\hat{ }ΔCt) |
| AML14 | AML | M2 | 15 | 9.2 | t(8:21) | Alive | 0.004800831 |
| AML66 | AML | M2 | 7 | 33.1 | Normal | Alive | N/A |
| AML67 | AML | M2 | 9 | 3.6 | Abnormal | Alive | 0.008708661 |
| AML68 | AML | M1 | 5 | 286 | Normal | Alive | 0.186856156 |
| AML12 | AML | M2 | 11 | 3.8 | Normal | Alive | N/A |
| AML24 | AML | M3 | 9 | 86 | t(15:17) | Alive | N/A |

### Example 5

### Use of Fat1 as a Therapeutic Target

Using an in vitro cell line panel, Fat1 was found to be expressed at the protein level in three out of the four T-ALLs (Jurkat, JM and Molt-4), both preB-ALL cell lines (LK63 and Nalm-6) and one AML cell line (THP-1). Consistent with this analysis, examination of microarray data from clinical specimens confirmed the significant presence of Fat1 transcript in 63.5% of T-ALL, 29% of B-ALL and 11% of AML cases, thereby demonstrating that Fat1 expression in leukemia was not an artifact of cell culture. Furthermore, there was no detectable Fat1 expression in peripheral blood mononuclear cells, either at the transcript level by qPCR or at the protein level by Western blotting. In silico analysis also showed no Fat1 expression in 38 hematopoietic cell line precursors, with the exception of early and late erythroid lineage signatures. The expression of Fat1 on a significant proportion of leukemic but not normal, non-leukemic hematopoietic cells highlights Fat1 as a therapeutic target in the treatment of leukemia.

This study also shows that in two independent genome-wide array data sets from matched pediatric preB-ALL diagnosis-relapse samples, Fat1 expression was an independent prognostic marker, whereby high Fat1 expression at diagnosis predicted poor outcome. The use of matched pairs has a number of distinct advantages, especially the fact that each patient acts as its own control. Moreover, these cohorts provide important information on the genetic changes and biological mechanisms that occur in preB-ALL relapse.

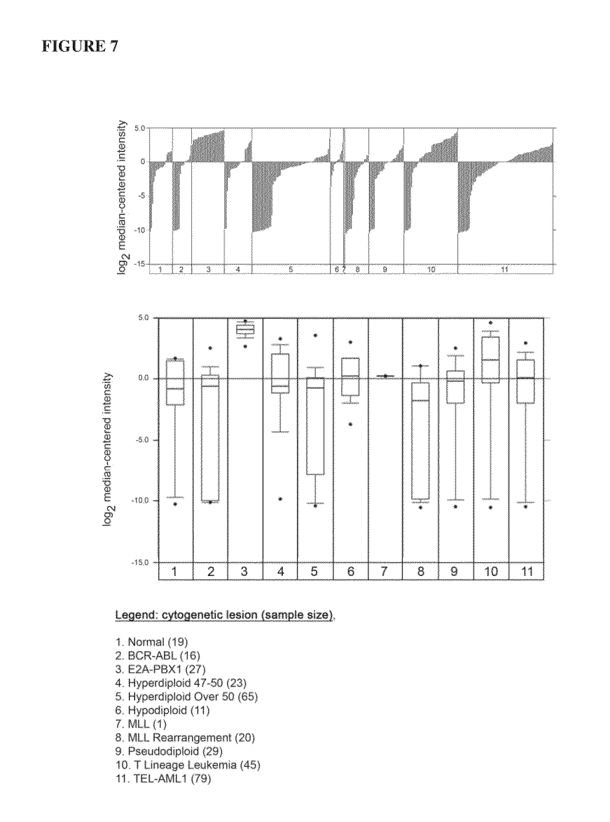

The emergence of resistance and prognostic value of any factor is directly associated with the type of treatment delivered. In the present study, high Fat1 expression is associated with earlier relapse and appears not to be associated with treatment. These findings demonstrate that Fat1 is an independent prognostic marker in paired diagnosis-relapse patients and thus yields important information behind the biology of relapse, information that continues to be needed to successfully cure the patients whom relapse. Further, by verifying that Fat1 is abnormally expressed in leukemia cells compared with normal blood cells, evidence is provided for Fat1 as a bona fide target for therapy and a target that may help overcome some of the mechanisms by which resistant lympho-blasts evade cytotoxicity. Moreover, given the frequency of Fat1 expression in a range of phenotypically diverse leukemias, concomitant with little or no expression on normal, non-leukemic peripheral blood cells and their early hematopoietic progenitors, Fat1 is an ideal target for the novel therapeutics, including antibody-based therapeutics. Whilst not limiting their application, such therapeutics may be of particular benefit to those patients with leukemia who also carry a cytogenetic lesion incorporating a translocation between chromosome 1 and 19 (t(1:19)), examples of which are shown in FIGS. 6 and 7.

### REFERENCES

Bhojwani D, et al. Blood 2006; 108: 711-717
Cox B, et al. Dev Dyn 2000; 271:233-240
Eckfeldt C E, et al. PLoS Biol 2005; 3: e254
Katoh Y, et al. Int J Mol Med 2006; 18:523-528
Ponassi M, et al. Mech Dev 1999; 80:207-212
Sadeqzadeh E, et al. J Biol Chem 2011; 286: 28181-28191
Settakorn J, et al. J Clin Pathol 2005; 58:1.249-1254
Staal F J, et al. Leukemia 2010; 24: 491-499
Valk P J, et al. N Engl Med 2004; 350: 1617-1628

1. A composition comprising an agent that is selectively cytotoxic to leukemic cells or neoplastic precursors thereof that express Fat1, or a homolog of Fat1 that is substantially not expressed on normal blood cells, and one or more pharmaceutically acceptable carriers, diluents or excipients.

2. The composition of claim 1, wherein the agent is an antibody or Fat1-binding fragment thereof.

3. The composition of claim 2, wherein the antibody is a monoclonal antibody or a Fat1-binding fragment thereof.

4. The composition of claim 3, wherein the monoclonal antibody is a human antibody or a humanized or deimmunized form of a non-human antibody.

5. The composition of claim 1, wherein the agent is a multi-specific antibody, which binds to at least two antigens on the leukemic cells or their precursors, wherein at least one antigen is Fat1 or its homolog.

6. The composition of claim 5, wherein the multi-specific antibody is a bi-specific antibody that binds to Fat1 or its homolog.

7. The composition of claim 2 wherein the antibody or Fat1-binding fragment thereof is labeled with a cytotoxic moiety.

8. The composition of claim 2, wherein the antibody or Fat1-binding fragment thereof is cytotoxic to the cells by complement-directed means.

9. The composition of claim 1, wherein the leukemic cell is a B- or T-lineage cell.

10. The composition of claim 9, wherein the cell is an acute lymphoblastic leukemic cell.

11. The composition of claim 1, further comprising another anti-cancer agent.

12. A method for treating a subject with a leukemia, said method comprising administering to said subject an effective amount of a composition according to claim 1.

13. The method of claim 12, wherein the subject has been previously treated for leukemia or other cancer.

14. The method of claim 13, wherein the subject is in remission.

15. The method of claim 12, further comprising administering to the subject another anti-cancer agent.

16. A method for the diagnosis or prognosis of a leukemia in a subject, the method comprising analyzing a blood sample from the subject for the presence of cells that express Fat1 or a homolog thereof that is substantially not expressed on normal blood cells, wherein the presence of a cell that expresses Fat1 or its homolog provides an indication of the presence of a leukemic cell or a precursor thereof.

17. The method of claim 16, wherein the analyzing step comprises contacting a primary binding agent that is capable of specifically binding to Fat1 on a blood cell, wherein the binding of the primary binding agent to the cell is indicative of presence of a cell that expresses Fat1 or its homolog.

18. The method of claim 17, wherein the primary binding agent is labeled with a detectable label.

19. The method of claim 17, wherein binding of the primary binding agent to the cell is detected by binding of a secondary binding agent that specifically binds to the primary binding agent, wherein the secondary binding agent is labeled with a detectable label.

20. The method of claim 16, wherein the leukemic cell is a B- or T-lineage cell.

21. The method of claim 20, wherein the cell is an acute lymphoblastic leukemic cell.

22. The method of claim 16, wherein the analyzing step comprises contacting nucleic acid from the blood sample with an oligonucleotide probe that is capable of hybridizing to a nucleic acid sequence encoding Fat1, or a homolog thereof, wherein the binding of the probe to the nucleic acid from the blood sample is indicative of presence of a cell that expresses Fat1 or its homolog.

23. A therapeutic protocol for treating leukemia in a subject, said protocol comprising the steps of:
(a) performing the method of claim 16 to determine the presence in said subject of cells that express Fat1 or a homolog thereof that is substantially not expressed on normal blood cells;
(b) administering to a subject who contains Fat1-expressing cells an agent that is selectively cytotoxic to leukemic cells, or neoplastic precursors thereof that express Fat1 or a homolog of Fat1 that is substantially not expressed on normal blood cells;
(c) monitoring for a reduction in the presence of Fat1-expressing cells over time;
wherein a reduction in Fat1-expressing cells over a period of time is indicative of a successful treatment.

24. The therapeutic protocol of claim 23, wherein the analyzing step comprises contacting a primary binding agent that is capable of specifically binding to Fat1 on a blood cell, wherein the binding of the primary binding agent to the cell is indicative of presence of a cell that expresses Fat1 or its homolog.

25. The therapeutic protocol of claim 23, wherein the analyzing step comprises contacting nucleic acid from the blood sample with an oligonucleotide probe that is capable of hybridizing to a nucleic acid sequence encoding Fat1, or a homolog thereof, wherein the binding of the probe to the nucleic acid from the blood sample is indicative of presence of a cell that expresses Fat1 or its homolog.

26. A method of vaccinating a subject against leukemia, the method comprising administering to the subject an amount of a compound comprising a Fat1 polypeptide, or a immunogenic fragment thereof, effective to stimulate antibodies against Fat1 expressed by cells in the subject.
